(19) United States
(12) Patent Application Publication
(10) Pub. No.: US 2026/0248524 A1
(43) Pub. Date: Aug. 27, 2026

(54) Clot Retrieval Device

(71) Applicant: Terumo Corporation, Tokyo (JP)

(72) Inventors: Anup Dasnurkar, Tokyo (JP); Maricela Walker, Tokyo (JP); Jake Doucet, Tokyo (JP)

(21) Appl. No.: 19/139,817

(22) PCT Filed: Dec. 21, 2023
(86) PCT No.: PCT/US2023/085555

§ 371 Date: Jun. 16, 2025

Related U.S. Application Data
(60) Provisional application No. 63/476,610, filed on Dec. 21, 2022.

(51) Int. Cl. A61B 17/221; A61B 17/3207

(52) U.S. Cl. CPC A61B 17/221; A61B 17/320725; A61B 2017/2212; A61B 2017/2215

(57) ABSTRACT

Clot retrieval devices are described that may include a cutting element and a clot capture basket connected to a distal region of an elongated device body and located proximally or distally of each other. The cutting element may help remove or cut a clot from a vessel wall and/or may help cut or breakup a clot into smaller components/pieces. The capture basket may capture at least some of the clot (e.g., whole or smaller pieces) and withdraw the clot/pieces from the vessel. The device body may include one component or several different components that may move relative to each other to assist in the cutting and capture process.

### RELATED APPLICATIONS

This application claims the benefit of and priority to U.S. Provisional Application Ser. No. 63/476,610, filed Dec. 21, 2022, and entitled Clot Retrieval Device, which is hereby incorporated herein by reference.

### BACKGROUND

Blood clots typically include platelets, fibrinogen, and/or other clotting proteins that form a mass within a blood vessel. Thrombosis typically refers to a blood clot that is lodged or fixed to an interior of a vessel and a thromboembolism typically refers to a blood clot that has dislodged from one location and travels around a circulatory system, often to block one or more vessels elsewhere in a patient.

A variety of different medical conditions may result from obstruction of blood, depending on where the blood clot migrates to. For example, obstruction of vessels near or within the brain may result in strokes, while obstruction of vessels in or near the lungs may result in pulmonary embolisms. For that reason, it is desirable to treat blood clots before serious medical conditions arise.

Clots may be treated in different ways, including with medications, balloon angioplasty, aspiration, clot retrieval, or removal devices, and combinations thereof. Hence, there is an ever-present need to improve the treatment and removal of blood clots from the vasculature of a patient.

### SUMMARY

The present specification is generally directed to clot retrieval devices and methods of use thereof.

In some aspects, the techniques described herein relate to a clot removal device, including: an elongated device body having a distal region; a cutting element located at the distal region of the elongated device body; and, a capture element located at the distal region of the elongated device body; wherein the capture element has a radially compressed shape and a radially expanded shape forming a cavity having an opening into the cavity.

In some aspects, the techniques described herein relate to a clot removal device, wherein the cutting element includes a wire or ribbon having at least one edge extending along at least part of the cutting element.

In some aspects, the techniques described herein relate to a clot removal device, wherein the elongated device body includes an elongated inner catheter member positioned within a lumen of an elongated outer catheter member; and wherein the elongated inner catheter member can move relative to the elongated outer catheter member.

In some aspects, the techniques described herein relate to a clot removal device, wherein the capture element is connected to a distal region of the elongated inner catheter member and the cavity of the capture element opens in a proximal direction; and wherein the cutting element is connected between a distal region of the elongated outer catheter member and the capture element.

In some aspects, the techniques described herein relate to a clot removal device, wherein the cutting element is one or a plurality of wires; and wherein the wires include one or more sharpened edges.

In some aspects, the techniques described herein relate to a clot removal device, wherein the capture element is connected to a distal region of the outer catheter member and the cavity of the capture element opens in a distal direction.

In some aspects, the techniques described herein relate to a clot removal device, wherein the cutting element is connected to the distal region of the outer catheter member and to a distal region of the inner catheter member; wherein the cutting element includes one or a plurality of wires with sharpened edges.

In some aspects, the techniques described herein relate to a clot removal device, further including a radially expandable closure member; wherein the expandable closure member has an expanded configuration sized to block the opening of the cavity.

In some aspects, the techniques described herein relate to a clot removal device, wherein the outer catheter member includes a proximal aspiration port for connection to an aspiration source.

In some aspects, the techniques described herein relate to a clot removal device, wherein the capture element is a mesh basket and the opening into the cavity is proximally-facing.

In some aspects, the techniques described herein relate to a clot removal device, wherein the cutting element is disposed around at least part of an edge of the opening into the cavity.

In some aspects, the techniques described herein relate to a clot removal device, including: an elongated device body having an elongated inner catheter member positioned within a lumen of an elongated outer catheter member; and wherein the elongated inner catheter member is movable relative to the elongated outer catheter member; at least one wire connected at a distal region of the elongated inner catheter member and at a distal region of the elongated outer catheter member; and, an expandable capture basket having a radially compressed shape and a radially expanded shape forming a cavity sized to capture a clot.

In some aspects, the techniques described herein relate to a clot removal device, wherein the at least one wire has an expanded shape extending away from the elongated inner member.

In some aspects, the techniques described herein relate to a clot removal device, wherein the at least one wire is connected to the expandable capture basket such that when the elongated inner catheter member is moved distally relative to the elongated outer catheter member, the at least one wire substantially closes a proximally-facing opening of the cavity.

In some aspects, the techniques described herein relate to a clot removal device, wherein the at least one wire helically encircles the elongated inner member.

In some aspects, the techniques described herein relate to a clot removal device, wherein a proximal portion of the at least one wire is positioned within the cavity of the expandable capture basket and is positioned out a distally-facing opening of the cavity.

In some aspects, the techniques described herein relate to a clot removal device, further including a mesh shield having a radially expanded shape; and wherein the elongated inner member is proximally movable to move the mesh shield adjacent to the distally-facing opening of the cavity.

In some aspects, the techniques described herein relate to a clot removal device, further including a first radiopaque marker at the distal region of the elongated inner catheter member and a second radiopaque marker at the distal region of the elongated outer catheter member.

In some aspects, the techniques described herein relate to a clot removal device, wherein the elongated inner catheter member is rotatable relative to the elongated outer catheter member such that the at least one wire is wrapped or unwrapped around the elongated inner catheter member.

In some aspects, the techniques described herein relate to a clot removal device, including: an elongated device body; a cutting element means for cutting a clot; and, a capture element means for capturing a clot after the cutting element means has cut the clot.

In some aspects, the techniques described herein relate to a clot removal device, including: an elongated device body; a cutting element connected at a distal end of the elongated device body; the cutting element including a plurality of struts connected together in a tubular shape that has a radially compressed configuration and a radially expanded configuration; wherein at least some of the plurality of struts have one or more cutting surfaces at a proximal portion of the cutting element.

In some aspects, the techniques described herein relate to a clot removal device, wherein at least some of the plurality of struts are connected together in a V shape or an alternating wave shape.

In some aspects, the techniques described herein relate to a clot removal device, wherein at least some of the plurality of struts are tapered in width, where the width of the at least some of the plurality of struts is relatively wider 1) at a proximal region of the cutting element, 2) at a distal region of the cutting element, 3) at a middle region of the cutting element, or 4) at the proximal region and the distal region of the cutting element relative to the middle region of the cutting element.

In some aspects, the techniques described herein relate to a clot removal device, wherein the cutting surfaces are scalloped, angled, or sloped regions along a strut.

In some aspects, the techniques described herein relate to a clot removal device, further including a wire connected to the elongated device body and to a proximal portion of the cutting element.

In some aspects, the techniques described herein relate to a clot removal device, wherein the wire is connected via one or more of the following: 1) through apertures in at least some of the plurality of struts, 2) by being tied around at least some of the plurality of struts, 3) by being welded to at least some of the plurality of struts, or 4) by being adhered with adhesive to at least some of the plurality of struts.

In some aspects, the techniques described herein relate to a clot removal device, further including a capture element connected to the cutting element.

In some aspects, the techniques described herein relate to a clot removal device, wherein the capture element is connected to a distal portion of the cutting element.

In some aspects, the techniques described herein relate to a clot removal device, wherein the capture element is a basket including a braided mesh or a membrane.

In some aspects, the techniques described herein relate to a clot removal device, including: an elongated device body; and, a cutting element connected at a distal end of the elongated device body; the cutting element including a tubular shape that has a radially compressed configuration and a radially expanded configuration; wherein a proximal end of the tubular shape includes a loop positioned at a biased angle within an inclusive range of 90 degrees to 180 degrees relative to a longitudinal axis of the tubular shape; and wherein the loop includes one or more cutting surfaces.

In some aspects, the techniques described herein relate to a clot removal device, wherein the loop is a closed loop or an open loop.

In some aspects, the techniques described herein relate to a clot removal device, wherein the one or more cutting surfaces are located along one or more of 1) an inner surface of the loop, 2) an outer surface of the loop, or 3) a side surface of the loop.

In some aspects, the techniques described herein relate to a clot removal device, wherein the one or more cutting surfaces are positioned at a single angle relative to the longitudinal axis of the tubular shape or at multiple angles relative to the longitudinal axis of the tubular shape.

In some aspects, the techniques described herein relate to a clot removal device, wherein the one or more cutting surfaces are smooth or are serrated.

In some aspects, the techniques described herein relate to a clot removal device, wherein the tubular shape further includes a plurality of struts connected distally to the loop and that form a plurality of cells.

In some aspects, the techniques described herein relate to a clot removal device, further including an elongated strut extending proximally from the loop.

In some aspects, the techniques described herein relate to a clot removal device, further including an elongated strut extending proximally from a distal-most position of the loop.

In some aspects, the techniques described herein relate to a clot removal device, including: an elongated device body; a cutting element located at a distal region of the elongated device body; the cutting element including a tubular shape that has a radially compressed configuration and a radially expanded configuration; the cutting element having a proximal region including one or more cutting surfaces; a capture element located distally of the cutting element and forming a cavity opening towards the cutting element; and, a distal tip located distally of the capture element.

In some aspects, the techniques described herein relate to a clot removal device, wherein the distal tip is connected to a distal end of the elongated device body.

In some aspects, the techniques described herein relate to a clot removal device, further including a guidewire lumen extending through the elongated device body and through the distal tip.

In some aspects, the techniques described herein relate to a clot removal device, wherein the capture element is connected to the cutting element and to the distal tip.

In some aspects, the techniques described herein relate to a clot removal device, wherein the capture element is a polymer film or a polymer fabric forming the cavity.

In some aspects, the techniques described herein relate to a clot removal device, wherein the polymer film or polymer fabric are composed of nylon, Pebax, PET, or polyurethane.

In some aspects, the techniques described herein relate to a clot removal device, wherein the polymer film or polymer fabric are blood permeable.

In some aspects, the techniques described herein relate to a clot removal device, wherein the capture element further includes a braided or laser-cut structure positioned around the polymer film or polymer fabric.

In some aspects, the techniques described herein relate to a clot removal device, including: an elongated device body; and, a cutting and capture element located at a distal region of the elongated device body; the cutting and capture element including a tubular shape that has a radially compressed configuration and a radially expanded configuration; wherein the tubular shape has a proximal portion including a proximal opening and one or more cutting surfaces; and wherein the tubular shape has a distal portion forming a cavity with a plurality of blood-permeable openings.

In some aspects, the techniques described herein relate to a clot removal device, wherein the proximal portion of the tubular shape includes a braided or laser-cut structure forming a plurality of open cells.

In some aspects, the techniques described herein relate to a clot removal device, wherein the cavity is further formed by a layer of 1) a film, 2) a fabric, or 3) a braided mesh.

In some aspects, the techniques described herein relate to a clot removal device, wherein the film, the fabric, or the braided mesh are composed of polyester.

In some aspects, the techniques described herein relate to a clot removal device, wherein the film, the fabric, or the braided mesh are located on an inside of the proximal portion of the tubular shape or on an outside of the proximal portion of the tubular shape.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. Reference is being made to the accompanying drawings, in which:

FIG. 1 illustrates a side view of a clot retrieval device at a respective position during the operation of the clot retrieval device, according to one example of the present disclosure.

FIG. 2 illustrates a side view of the clot retrieval device of FIG. 1 at a respective position during the operation of the clot retrieval device, according to one example of the present disclosure.

FIG. 3 illustrates a side view of the clot retrieval device of FIG. 1 at a respective position during the operation of the clot retrieval device, according to one example of the present disclosure.

FIG. 4 illustrates a side view of the clot retrieval device of FIG. 1 at a respective position during the operation of the clot retrieval device, according to one example of the present disclosure.

FIG. 5 illustrates a side view of the clot retrieval device of FIG. 1 at a respective position during the operation of the clot retrieval device, according to one example of the present disclosure.

FIG. 6 illustrates a side view of another example of a clot retrieval device, according to one example of the present disclosure.

FIG. 7 illustrates a side view of the clot retrieval device of FIG. 6 at a respective position during the operation of the clot retrieval device, according to one example of the present disclosure.

FIG. 8 illustrates a side view of the clot retrieval device of FIG. 6 at a respective position during the operation of the clot retrieval device, according to one example.

FIG. 9 illustrates a side view of another example of a clot retrieval device, according to one example of the present disclosure.

FIG. 10 illustrates a side view of the clot retrieval device of FIG. 9 at a respective position during the operation of the clot retrieval device, according to one example of the present disclosure.

FIG. 11 illustrates a cross-sectional view of a cutting element having a rectangular cross section, according to one example of the present disclosure.

FIG. 12 illustrates a cross-sectional view of a cutting element having a generally rectangular cross section and a pointed or triangular end, according to one example of the present disclosure.

FIG. 13 illustrates a cross-sectional view of a cutting element having a generally rectangular or hexagonal cross section and two pointed or triangular ends, according to one example of the present disclosure.

FIG. 14 illustrates a cross-sectional view of a cutting element having a generally rectangular cross-sectional shape that is bent or curved, or might otherwise be considered concave or convex, according to one example of the present disclosure.

FIG. 15 illustrates a cross-sectional view of a cutting element with a generally rectangular cross-sectional shape with one or more pointed or triangular portions along a side, according to one example of the present disclosure.

FIG. 16 illustrates a cross-sectional view of a cutting element with a generally circular cross-section shape with one or more pointed or triangular portions along a side, according to one example of the present disclosure.

FIG. 17 illustrates a cross-sectional view of a cutting element with a generally star cross-sectional shape with a plurality of pointed or triangular portions, according to one example of the present disclosure.

FIG. 18 illustrates a side view of a cutting element, according to one example of the present disclosure.

FIG. 19 illustrates a side view of the cutting element in FIG. 18 and a wire, according to one example of the present disclosure.

FIG. 20 illustrates a top view of another example of a cutting element in a radially expanded configuration, according to one example of the present disclosure.

FIG. 21 illustrates a side view of the cutting element of FIG. 20 in the radially expanded configuration, according to one example of the present disclosure.

FIG. 22 illustrates a cross-section of the cutting element of FIGS. 20 and 21, according to one example of the present disclosure.

FIG. 23 illustrates a cross-section of the cutting element of FIGS. 20, 21, and 22, according to one example of the present disclosure.

FIG. 24 illustrates a cross-section of the cutting element of FIGS. 20, 21, 22, and 23, according to one example of the present disclosure.

FIG. 25 illustrates a side view of another example of a cutting element in a radially compressed configuration, according to one example of the present disclosure.

FIG. 26 illustrates a top view of the cutting element of FIG. 25 in the radially compressed configuration, according to one example of the present disclosure.

FIG. 27 illustrates a cross-section of the cutting element of FIGS. 25 and 26 in a radially expanded configuration, according to one example of the present disclosure.

FIG. 28 illustrates a cross-section of the cutting element of FIGS. 25, 26, and 27, according to one example of the present disclosure.

FIG. 29 illustrates a side view of another example of a clot removal device, according to one example of the present disclosure.

FIG. 30 illustrates a top view of the clot removal device of FIG. 29 in a deployed state, according to one example of the present disclosure.

FIG. 31 illustrates a side view of the clot removal device of FIGS. 29 and 30 in a retracted state, according to one example of the present disclosure.

FIG. 32 illustrates a side view of another example of a clot removal device, according to one example of the present disclosure.

FIG. 33 illustrates a side view of another example of a clot removal device, according to one example of the present disclosure.

FIG. 34 illustrates a side view of a cutting and capture element of the clot removal device of FIG. 33, according to one example of the present disclosure.

FIG. 35 illustrates a side view of a cutting element in a radially expanded configuration, according to one example of the present disclosure.

FIG. 36 illustrates a bottom view of the cutting element of FIG. 35 in the radially expanded configuration, according to one example of the present disclosure.

FIG. 37 illustrates a top view of the cutting element of FIGS. 35 and 36, according to one example of the present disclosure.

FIG. 38 illustrates a side view of a first serrated cutting element, according to one example of the present disclosure.

FIG. 39 illustrates a side view of the first serrated cutting element of FIG. 38, according to one example of the present disclosure.

FIG. 40 illustrates a side view of a second serrated cutting element, according to one example of the present disclosure.

FIG. 41 illustrates a side view of the second serrated cutting element of FIG. 40, according to one example of the present disclosure.

FIG. 42 illustrates a side view of a third serrated cutting element, according to one example of the present disclosure.

FIG. 43 illustrates a side view of the third serrated cutting element of FIG. 42, according to one example of the present disclosure.

FIG. 44 illustrates a side view of a fourth serrated cutting element, according to one example of the present disclosure.

FIG. 45 illustrates a side view of the fourth serrated cutting element of FIG. 44, according to one example of the present disclosure.

FIG. 46 illustrates a cutting element defining a pair of sharpened edges, according to one example of the present disclosure.

FIG. 47 illustrates a cutting element defining a single sharpened edge, according to one example of the present disclosure.

FIG. 48 illustrates a cross-section of a double-edged cutting element, according to one example of the present disclosure.

FIG. 49 illustrates a cross-section of a double bevel cutting element, according to one example of the present disclosure.

FIG. 50 illustrates a convex cutting element having a pair of outwardly curved cutting edges, according to one example of the present disclosure.

FIG. 51 illustrates a concave cutting element having a pair of inwardly curved cutting edges, according to one example of the present disclosure.

FIG. 52 illustrates a single-edged cutting element having an angled cutting edge, according to one example of the present disclosure.

FIG. 53 illustrates a combination cutting element, according to one example of the present disclosure.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

The terms blood clot or clot are used in this specification and may include a mass within a blood vessel that may be composed of platelets, fibrinogen, and/or other clotting proteins. These terms may include thrombus, embolus, thromboembolus, obstruction, blockage, or similar terms and variation.

Generally, clots may be relatively firm or hard, or relatively soft or semi-liquid, depending upon their compositions. Typically, soft clots tend to be relatively small and tend to travel through the arteries of a vascular system. Firm clots are more likely to be found in veins, are often relatively longer, and are often attached to the wall of the vein. For example, some firm clots may be as large as 30-40 cm in size. Further, depending on the location of a firm clot, it can be partially or heavily calcified. Deep vein thrombosis (DVT) is one known condition that may occur when a clot, often a firm clot, develops in a vein. Most DVT occurs in a patient's lower legs, though occurrences in the arm, brain, intestines, liver, or kidney are also possible.

Since DVT sometimes involves relatively long, firm clots that are attached to a wall of a vein, treatment typically includes the use of anticoagulants (i.e., blood thinners), compression stockings to help reduce swelling and blood pooling, balloon angioplasty to crush the clot and help open the vein, and stent placement to help create an open passage through the clot. The aggressiveness of treatment may sometimes depend on the location of the clot in the legs. For example, clots below the knee are often treated less aggressively (e.g., with less interventional procedures) relative to clots in above the knee locations.

While clot retrieval devices are typically used for softer clots, such as those commonly found in arteries in or near the brain, or the lungs, these devices often have more difficulty capturing firmer clots. Soft clots tend to be relatively short, and are often unattached to vessel walls, which allows capture devices to easily deploy, encompass, and withdraw the clot. And, since soft clots tend to be located within arteries, clot retrieval devices are typically constructed to be soft and gentle on the arterial walls to help reduce the likelihood of an arterial rupture.

However, due to the generally longer length, firm structure, and relatively strong attachment to vessel walls, firm clots (e.g., DVT clots within a patient's legs) may be difficult to treat with many known clot retrieval devices. For example, existing arterial clot retrieval devices may be too gentle, or otherwise unable, to easily remove firm clots from their relatively strong attachment or fixation to vessel walls. This may particularly be the case for longer clots, where multiple cutting passes of a retrieval device may be necessary (e.g., a retrieval device may need to be deployed within a firm clot several times to progressively remove sections of the clot).

The present specification includes clot retrieval devices that may be particularly helpful in removing clots that are relatively firm, relatively long, attached to a vessel wall, and/or are calcified (e.g., clots associated with DVT, either above or below the knee). While the term clot retrieval device, or clot removal device, may be primarily used herein, this term may also include thrombectomy devices, atherectomy devices, or similar terminology.

Generally, the examples of this specification may include a cutting element and a clot capture element (also referred to as a capture basket) connected to a distal region of an elongated device body and located proximally or distally of each other (e.g., longitudinally adjacent or near each other). The cutting element may help remove or cut a clot from a vessel wall and/or may help cut or breakup a clot into smaller components or pieces. The capture basket may capture at least some of the clot (e.g., whole or smaller pieces) and withdraw the clot, or pieces thereof, from the vessel. As described further below, the elongated device body may include one component or several different components that may move relative to each other.

The capture basket may include a structure having a radially compressed state or configuration and a radially expanded state or configuration. In the radially expanded state or configuration, the capture basket may include an opening into a space, cavity, or lumen within the capture basket. The opening may be sized to allow clots or pieces of clots to pass through. The opening may be oriented proximally, distally, or sideways (e.g., orthogonally or perpendicular) relative to an axis of the device, or alternatively at various orientations therebetween, such as, but not limited to, at a 45 degree angle, or at a 315 degree angle, relative to an axis of the device, to thereby create a biased (e.g., non-orthogonal or perpendicular) cut opening.

In some examples, the capture basket may be formed from, or may connected to, a variety of different components that allow it to expand from its radially compressed state to its radially expanded state. For example, the capture basket may be formed from a plurality of braided wires or filaments. The wires or filaments may be composed of a shape memory alloy (e.g., Nitinol wires), non-shape memory alloy (e.g., stainless steel wires), or polymer filaments or wires (e.g., PET filaments). In another example, the capture basket may be formed from a framework (e.g., Nitinol wire or a laser cut Nitinol integral structure) that may optionally have braided wires, filaments (e.g., both as previously described), or a membrane connected to and disposed over the structure.

In another example, the capture basket may be an integral laser cut or machined mesh structure that may optionally have a membrane, such as an inner membrane layer or inner liner, connected to and positioned within a cavity of the mesh structure, and/or an outer membrane layer, connected to and extending around the mesh structure. In further example, the capture basket and a cavity for receiving clots therein may be entirely composed of a membrane, a polymer film, or a polymer fabric, such as, but not limited to, nylon, polyether block amide (“Pebax”), polyethylene terephthalate (“PET”), or polyurethane. In a still further example, the capture basket may be representative of a capture area or length (e.g., cavity) of an integral laser cut or machined cutting and capture element that may optionally have a membrane, such as an inner membrane layer or inner liner, positioned within the capture area or element of the integral cutting and capture structure, and/or an outer membrane layer, connected to and extending around the integral cutting and capture structure.

The capture basket may self-expand from its radially compressed state to its radially expanded state due to, for example, one or more of its components being composed of a shape memory material with an expansion-driving memory shape, or by virtue of connection to a self-expanding cutting element. In another example, the capture basket may self-expand by virtue of its integral formation with a self-expanding cutting element. The capture basket may alternatively expand from its radially compressed state to its radially expanded state by manual actuation, such as via the use of one or more control wires that force or prop open the capture basket directly, or force or prop open a cutting element connected to the capture basket.

In some examples, the capture basket may also include a closure mechanism that reduces the size of, or even completely closes, the opening of the capture basket. For example, cutting elements, such as or one or more wires, may be connected or woven along part or all of the capture basket near its opening (e.g., near an edge forming the opening) to form a mesh. The one or more wires may be connected to the capture basket at single discrete locations or may be connected partially or fully around the opening. The one or more wires may be connected by adhesives, welding, weaving, braiding, knots, coils clamps, or other similar mechanisms or means. As the one or more wires is pulled relative to the capture basket, the capture basket cinches its opening partially or fully closed. In one example, the one or more wires may also be connected to a moveable component or member, such as an inner catheter member or an outer catheter member.

In another example, the capture basket may have a closure member, which also may be referred to as a shield or basket door, that is movable against the opening of the capture basket such that it partially or fully closes or obstructs the opening. In one example, the closure member may be a mesh structure similar to as described for the capture basket, and may self-expand (e.g., via a memorized and/or heat-set shape), or be manually expandable (e.g., via a control wire). The closure member may be moved against the opening by connecting each component to one of two different elongated components or structures of the elongated device body that may move axially or longitudinally relative to each other.

For example, one component may be, or be on, an outer catheter structure or tube and the other component may be, or be on, an inner catheter structure or member that moves into and out of the outer catheter structure (e.g., an outer tube or catheter having a lumen and an inner tube, rod, or wire sized to fit within the tube or lumen). In another example, one component may be on a first structure and the other component may be on an adjacent second structure (e.g., two tubes, rods, or wires that are adjacent and that may be located in an outer catheter). In another example, the capture basket may not have a closure mechanism beyond reducing or retracting the capture basket from its radially expanded state to at least partially into its radially compressed state and/or being withdrawn into a lumen of an outer catheter or sheath.

The clot retrieval device may include one, or a plurality of, cutting elements each having at least one cutting surface that may cut a clot from a vessel wall and/or cut a clot into smaller pieces. In that respect, portions of the cutting element or cutting elements including the cutting surface or surfaces may be at least partially located, or positioned, at a location spaced radially apart from an axis or cross-sectional middle of the elongated device body, such as the outer periphery near the position of a vessel wall.

In one example, the cutting element may include a wire, ribbon, or other similar elongated structures that have one or more surfaces or edges that may cut into a firm clot. For example, the cutting element may be a wire or ribbon, and may have a rectangular or square cross-sectional shape with four relatively sharp edges. In another example, the cutting element may have a rectangular or square cross-sectional shape with one side forming a triangular point (i.e., a triangular ridge extending along at least a portion of its length), or a rectangular or square cross-sectional shape with two sides that each form a triangular point (i.e., two triangular ridges extending along at least a portion of its length).

In another example, the cutting element may have a curved or substantially “C” shape, a circular cross-sectional shape with a triangular or pointed area (i.e., a triangular ridge extending along at least a portion of its length), or a cross sectional shape with a plurality of points (e.g., 3-10 triangular ridges extending along at least a portion of its length). Example diameters (in the case of wire shapes) or widths (in the case of ribbon shapes) may be within a range of about 0.001 mm to about 0.09 mm. In some examples, especially with some ribbon or laser cut flattened or elongated shapes, widths or wall thicknesses may be between about 0.1 mm and about 4 mm. The cutting element or cutting elements may be composed of a shape memory alloy such as Nitinol, a non-shape memory metal such as stainless steel or tantalum, or a polymer such as PET, among other materials.

The cutting element may take several different forms and positions on or along a clot retrieval device. In various examples, the cutting element may extend along a distal region of the clot retrieval device, connecting at proximal and/or distal locations in, or ends of, the distal region. In some examples, the cutting element, in an unrestrained expanded state, may be located helically around, or may radially encompass, at least a portion of the distal region and may at least partially expand radially outward from the elongated device body of the clot retrieval device. In some example, the cutting element, in an unrestrained expanded state, may alternatively extend linearly along the elongated body of the clot retrieval device and may at least partially expand radially outward from the elongated device body.

In the prior examples, the ends of the cutting elements may be fixed relative to each other such that their longitudinal position remains constant. In such examples, the cutting element may be connected such that, when unconstrained (e.g., via an outer catheter), it radially self-expands. Alternatively, each end of the cutting element may be connected to one of two components that move relative to each other, such as one end connecting to an outer catheter structure and the other end connecting to an inner catheter structure that moves into and out of the outer catheter structure (e.g., an outer tube and an inner tube, rod, or wire). In another example, one component may be on a first structure and the other component may be on an adjacent second structure (e.g., two tubes, rods, or wires that are side-by-side adjacent and that may be located in an outer catheter). In a further example, one component may be the outer catheter structure and the other may be a wire extending between a plurality of struts forming the cutting element and the inner catheter structure. Hence, the cutting elements of the present disclosure may be manually radially expanded from a radially compressed state or configuration to a radially expanded state or configuration, and/or twisted or untwisted, by a physician by moving the two movable components relative to each other (e.g., via proximal ends of the components).

In some examples, the cutting element may take the form of a partial or full loop that either self-expands, or manually expands such via operation of the moveable components, to a larger radial diameter (e.g., expands from a radially compressed state or configuration to a radially expanded state or configuration) when unconstrained. The partial or full loop of the cutting element may expand to a diameter either smaller than or similar to a target vessel size such that, when the clot retrieval device is moved, the loop cuts into the clot and either breaks it up or cuts it from a wall of the target vessel. The full or partial loop of the cutting element may be connected around an opening of a previously described capture basket, such that a sharp edge or cutting surface of the loop is oriented to cut into a clot as the clot retrieval device is pulled proximally towards it.

Alternatively, a full loop of the cutting element may be formed and supported by a plurality of laser cut, machined, or welded struts that may help expand the full loop of the cutting element, and position or locate it radially outward from a cross-sectional middle or the elongated device body of the clot retrieval device. In some such examples, the plurality of struts may define a tubular stent like structure formed entirely the plurality of struts, which in such examples, may include, but not limited to, 4, 6, 8, 10, 12, 14, 16, 18, 20, individual struts each connected to at least two adjacent struts. Further, in some such examples, several of the plurality of struts may each define a cutting surface or sharp edge oriented to cut into a clot as the clot retrieval device is pulled proximally towards it.

In further examples, a full or partial loop of the cutting element may be partially formed and support by a plurality of laser cut or machined struts that expand the full or portion loop, and position or locate it radially outward from a cross-sectional middle or the elongated device body of the clot retrieval device. In some such examples, the plurality of struts may extend distally over or around the elongated device body from a loop defining one or more cutting surfaces oriented to cut into a clot as the clot retrieval device is pulled proximally towards it. Further, in some such examples, the one or more cutting surfaces may be located along an inner surface of the loop, an outer surface of the loop, at one or more locations between the outer surface and the inner surface of the loop, along one or more side surfaces of the loop, or a combination thereof. In one such example, the one or more cutting surfaces may include a first cutting surface and an opposite and opposing second cutting surface each defined between an upper surface and a lower surface of the cutting element.

In still further examples, a full or partial loop of the cutting element may be partially formed and support by a plurality of laser cut or machined solid portions that may help expand the full or portion loop, and position or locate it radially outward from a cross-sectional middle or the elongated device body of the clot retrieval device. In some such examples, the one or more cutting surfaces may extend proximally from the full or partial loop toward or into a proximal portion of the cutting element, each of which are oriented to cut into a clot as the clot retrieval device is pulled proximally towards it. In some such examples, the one or more cutting surfaces may extend proximally from the loop, and may be located along an inner surface extending between a proximal and distal portion of the cutting element, along an outer surface extending between a proximal and distal portion of the cutting element, at one or more locations between the outer surface and the inner surface of the cutting element, along one or more side surfaces of the cutting element, or a combination thereof.

The cutting element may, in additional example, also take the form of several of the previously described forms, other forms described below. For example, a helical wire or ribbon and/or a loop may be included. The outer sheath of the clot retrieval device may be connected to a vacuum or aspiration source such that aspiration may be applied during part, or all, of the retrieval procedure. Again, any of the previously described features of the clot retrieval device may be mixed and matched together.

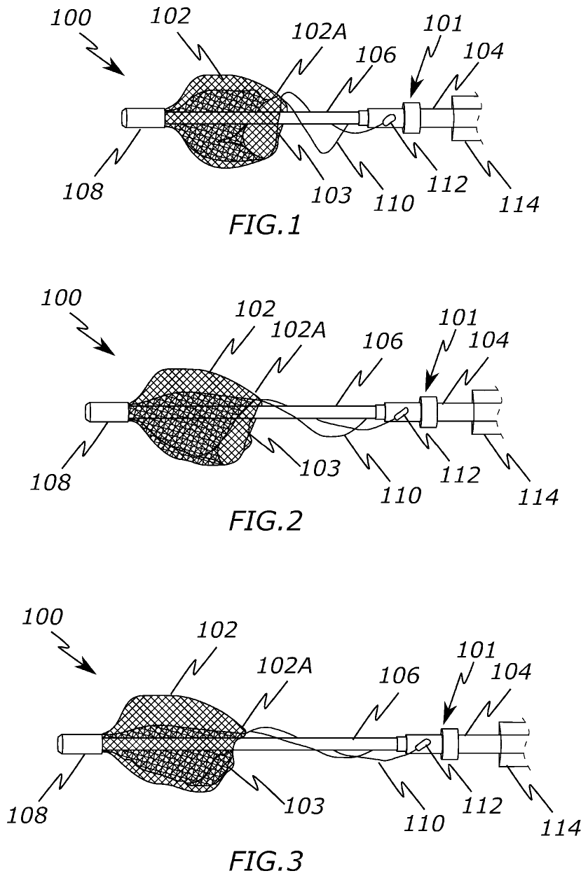

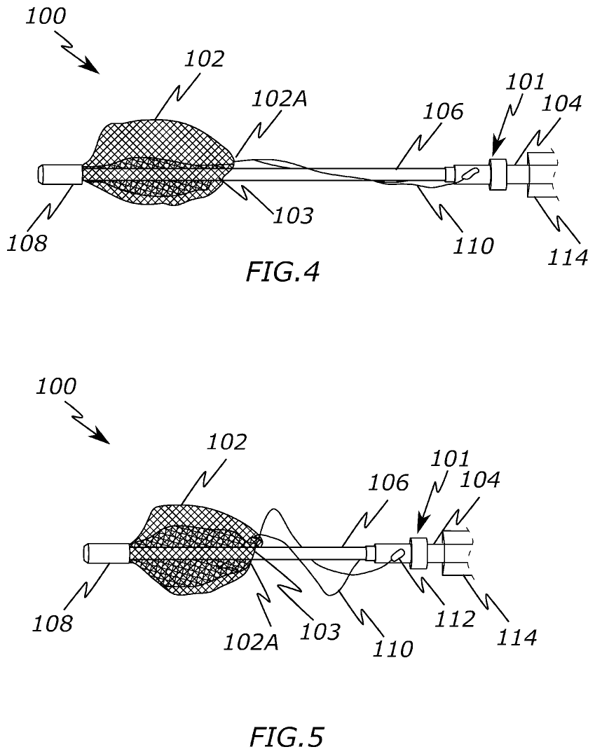

FIGS. 1-5 illustrate respective side views of an example clot retrieval device 100 at various positions or configurations to illustrate the closure of the interior of a capture basket 102 of the retrieval device 100 during operation. The clot retrieval device 100 may be used to cut or breakup a clot, and then capture and remove at least some portions, pieces, or components of the clot. More specifically, in some examples, the clot retrieval device 100 includes an elongated device body 101, a capture basket 102, and/or one or more wires 110.

FIG. 1 illustrates a first side view of a clot retrieval device 100. In some examples, the elongated device body 101 may be generally representative of a variety of different generally tubular or catheter-like components adapted for use during a clot removal procedure and is discussed with reference to various non-limiting examples of the present disclosure. For example, the elongated device body 101 may be representative of one or more tubular or otherwise hollow shafts including a lumen therein. In the illustrated example, the elongated device body 101 is an inner catheter member 106 positioned within a lumen of an outer catheter member 104. The inner catheter member 106 can move distally and/or proximally relative to the elongated outer catheter member 104. In some examples, the capture basket 102 may be composed of a mesh, such as braided from one or more wires composed of shape memory material (e.g., Nitinol). Alternatively, the capture basket 102 may have other forms or materials, such as, but not limited to, those described earlier. In the present example, the capture basket 102 may have an inner layer and an outer layer, such that the inner layer forms an inner cavity of the capture basket 102 and the outer layer forms an outer surface of the capture basket 102.

In some examples, the two layers (e.g., the inner layer and the outer layer) of the capture basket 102 may be formed from a single mesh tube that is folded back on itself, with its ends fixed to the clot retrieval device 100 via a tip or clamp member 108 (e.g., a metal or polymer tube or cap that is attached to the ends of the mesh tube and to the clot retrieval device 100 via adhesive, welding, and/or crimping means). Hence, the fold between the inner layer and the outer layer of the capture basket 102 may form a proximally facing edge 102A around an opening 103 of the capture basket 102. Alternatively, only a single layer mesh may be used to form the capture basket 102, such that a distal end of a single mesh tube is attached by the tip or clamp member 108, and a proximal end of the single mesh tube forms the proximally facing edge 102A.

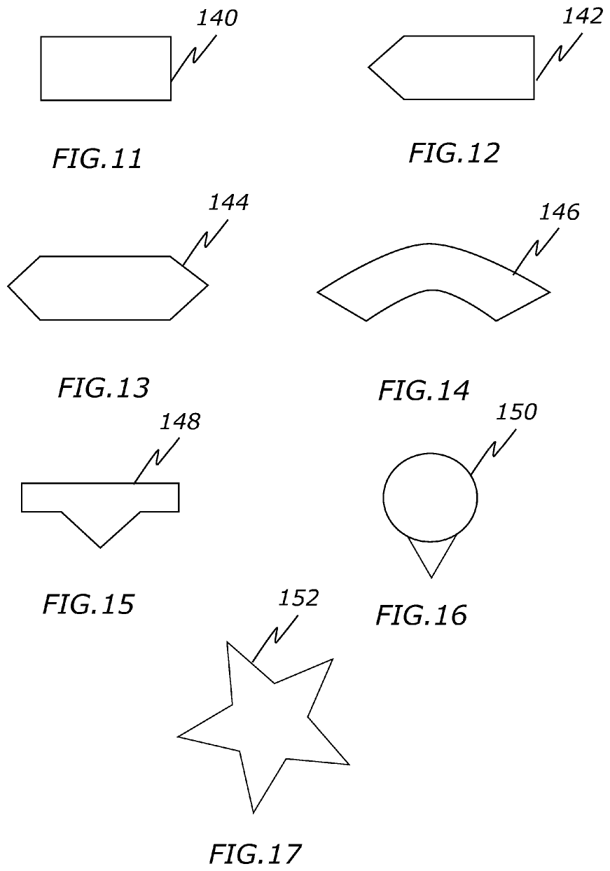

As previously discussed, in some examples, the clot retrieval device 100 includes at least one cutting element. In one example, the cutting element of the present disclosure may include the one or more wires 110. In the present example, the one or more wires 110 may be, or otherwise act, as a cutting element to cut into a clot. Note that while the term “wire” is generally used here, this term is meant to be inclusive of similar components such as, but not limited to, ribbons, or other thin and elongated components defining one or more relatively sharp edges along at least a portion of its body or outer surface, such as also discussed earlier, and further discussed below with reference to FIGS. 11-17, in this specification.

The one or more wires 110 may be composed of, for example, but not limited to, a shape memory alloy (e.g., Nitinol wires), a non-shape memory alloy, or a polymer. In the case of a shape memory alloy, the one or more wires 110 may be shape-set to form various shapes or configurations when unconstrained, such as a radially expanded helical shape extending around the distal region of the elongated device body 101 of the clot retrieval device 100. In various examples, the one or more wires 110 of the clot retrieval device 100 may include, but is not limited to, 1, 2, 3, 4, 5, 6, 7, 8, or other numbers of individual wires, ribbons, or relatively thin elongated bodies.

The one or more wires 110 may be manually controllable in their radial size and rotational position relative to the elongated device body 101, such as seen in FIGS. 1-5. This may be achieved by connecting the ends of each wire of the one or more wires 110 to components of the elongated device body 101 of the clot retrieval device 100 that move longitudinally or axially relative to each other. In the examples of the figures, such movable components of the elongated device body 101 may be an outer catheter member 104, and an inner catheter member 106 that may be moved at least partially into, and out of, the outer catheter member 104. A proximal end of each of the one or more wires 110 may be fixed or connected near a distal end of the outer catheter member 104, such as at location 112, via adhesives, welding, clamping (e.g., via one or more clamps), and/or other similar means or techniques.

In some examples, a distal end of each of the one or more wires 110 may be either directly connected to the inner catheter member 106 (e.g., via adhesives, welding, clamps, or similar techniques), or to another component connected to the inner catheter member 106. In one example, such as shown in FIGS. 1-5, the distal end of each of the one or more wires 110 is connected to the capture basket 102. Hence, outer catheter member 104 and the inner catheter member 106 may be moved relative to each other (e.g., rotated and/or longitudinally moved) to cause the one or more wires 110 to twist, untwist, radially expand, and/or radially compress. In that regard, the outer catheter member 104 and the inner catheter member 106 may extend to a proximal end of the clot retrieval device 100, and may include handles or similar proximal regions that a physician can move, rotate, or otherwise manipulate.

Additionally, the outer catheter member 104 and the inner catheter member 106 may be further located at least partially within an outer sheath 114 that both the outer catheter member 104 and the inner catheter member 106 may be advanced out of and retracted back into. Such an outer sheath 114 may serve to radially constrain any self-expanding components (e.g., the capture basket 102 or the one or more wires 110) and may at least partially recapture or receive the capture basket 102 after portions of a clot have been captured.

In some examples, the one or more wires 110 may be configured to close a cavity or interior of the capture basket 102. For example, the one or more wires 110 may be connected to the capture basket 102 such that, as the one or more wires 110 are tightened via movement of the inner catheter member 106 relative to the outer catheter member 104. When the one or more wires 110 are tightened, the one or more wires 110 can partially, or fully, cause the opening 103 of the capture basket 102 to close (e.g., shrink in diameter). This may be advantageous because the opening 103 of the capture basket 102 may be closed without substantially reducing the outer diameter of the remaining portion (e.g., a portion of the capture basket located distally to the one or more wires 110) of the capture basket 102. As may be appreciated, this may help the capture basket 102 to retain a clot, or clot pieces, within the capture basket 102 as the capture basket 102 and the one or more wires 110 are withdrawn into the outer sheath 114 during removal of the clot retrieval device 100 from the vasculature of a patient.

The closure of the interior of the capture basket 102 described above may be achieved in several ways. For example, the one or more wires 110 may be connected and/or woven within the mesh structure of the capture basket 102, such as at, or near, the proximally facing edge 102A. In an example where the one or more wires 110 include only a single wire, such a single wire may be tied, adhered, welded, clamped, or otherwise secured to one or more wires or ribbons of the mesh structure of the capture basket 102, and then woven into the mesh structure near, or at, the proximally facing edge 102A, such as either entirely around the opening 103 or partially around the opening 103 (e.g., three fourths of the distance around the opening) of the capture basket 102.

In an example where the one or more wires 110 include multiple wires, each of the multiple wires may be tied, adhered, welded, clamped, or otherwise secured to one or more wires of the mesh structure of the capture basket 102 near, or at, the proximally facing edge 102A. Depending on the individual number of wires the one or more wires 110 includes, only one connection to the mesh structure of the capture basket 102 may be sufficient, but alternatively, portions of each wire of the one or more wires 110 may also be woven through the mesh structure of the capture basket 102 at, or near, the proximally facing edge 102A.

FIGS. 1-5 show a progression of how the clot retrieval device 100 may be operated to cut and capture a clot. First, the clot retrieval device 100 may be positioned at a target location within vasculature of a patient in a variety of different ways, such as including, but not limited to, by following a guidewire (e.g., the inner catheter member 106 may include a guidewire lumen extending between its proximal and distal ends), by being advanced through a larger access catheter, or a combination thereof. In some examples, the outer sheath 114 may be used, and may be desirable for, capturing and removing a clot. In other examples, the outer sheath 114 may not be used or needed.

As shown in FIG. 1, once the distal region of the clot retrieval device 100 is positioned or located at a target location (i.e., near a clot), the distal region of the elongated device body 101 of the clot retrieval device 100 may be advanced through some, or all, of the clot (not shown). If the outer sheath 114 is used, both the inner catheter member 106 and the outer catheter member 104 can then be advanced distally out of the outer sheath 114 to, in turn, allow both the capture basket 102 and the one or more wires 110 to radially expand from a radially compressed shape or state to a radially expanded shape or state, such as seen in FIG. 1. The inner catheter member 106 and the outer catheter member 104 may be moved (e.g., translated axially or longitudinally and/or rotated) relative to each other to achieve a desired amount or degree of radial expansion, or a desired size and shape, of the one or more wires 110. For example, the one or more wires 110 may be formed to (e.g., expanded or moved into) a helical shape with a diameter that is about the same as a diameter of the opening 103 of the capture basket 102, an outer diameter of the capture basket 102, or the diameter of the target vessel (e.g., adjacent to the vessel wall).

Next, with the clot retrieval device 100 located at a position distal with respect the clot, the clot retrieval device 100 (or at least the inner catheter member 106 and the outer catheter member 104), may be moved proximally toward the clot so that the one or more wires 110 (e.g., one or more relatively sharp edges or surfaces of the one or more wires 110) cut into the clot. This may break off minor pieces of the clot, especially in cases of relatively long clots, may break off major pieces or most of the clot or cut the entire clot from the vessel wall. The clot retrieval device 100, or at least the inner catheter member 106 and the outer catheter member 104, may then be further moved proximally so that the clot enters the inner cavity or interior of the capture basket 102 through the opening 103.

FIGS. 2, 3, and 4 progressively illustrate how the opening 103 of the capture basket 102 may subsequently be closed by moving the inner catheter member 106 and the outer catheter member 104 relative to each other. For example, FIGS. 2-5 illustrates the outer catheter member 104 progressively being retracted proximally relative to the inner catheter member 106. As this occurs, the one or more wires 110 may proximally pull, and therefore close or cinch, the opening 103 of the inner cavity of the capture basket 102, and, in turn, capture the clot or clot pieces within the capture basket 102. Concurrently, the helical, tubular, or loop shape formed by the one or more wires 110 may decrease in diameter due to the proximal pulling of the capture basket 102.

As seen in FIG. 5, depending on how the one or more wires 110 are attached to the capture basket 102, the capture basket 102 may remain closed, even if the proximal pulling force from the outer catheter member 104 is released. For example, if the one or more wires 110 are woven at or near the proximally facing edge 102A of the capture basket 102, the pores or cells of the mesh structure of the capture basket 102 may close around the one or more wires 110 and creating friction therebetween. As may be appreciated, this friction may help maintain the opening 103 of the capture basket 102 in a closed state. Finally, the clot retrieval device 100 may be withdrawn from the patient, such as by first proximally retracting the inner catheter member 106 and the outer catheter member 104 of the elongated device body 101 at least partially back into the outer sheath 114, if the outer sheath 114 is used, and proximal removing the clot retrieval device 100 from the vasculature of the patient.

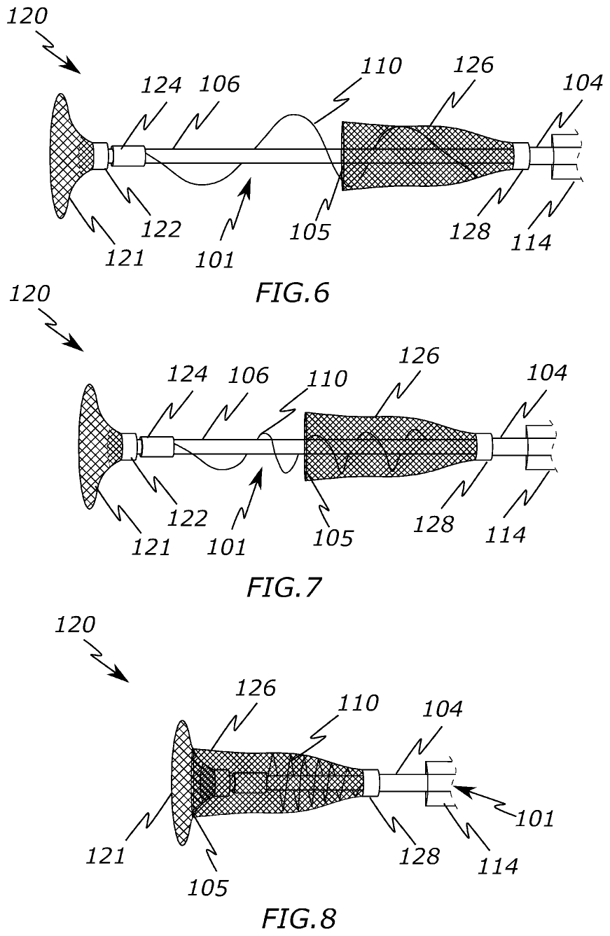

FIGS. 6-8 illustrate another example of a clot retrieval device 120. More specifically, FIGS. 6-8 illustrate an example progression of the clot retrieval device 120 cutting and capturing a clot (not shown). The clot retrieval device 120 may generally be similar to the clot retrieval device 100 previously described above with respect to FIGS. 1-5. However, in contrast to the clot retrieval device 100, the clot retrieval device 120 may include a capture basket 126 with a distally facing opening 105; and, a closure structure which includes a movable closure member 121 that may be moved against the distally facing opening 105 of the capture basket 126 to partially or fully close the distally facing opening 105 thereof.

In some examples, the capture basket 126 may be connected to the outer catheter member 104. For example, a proximal end of the capture basket 126 may be connected to the outer catheter member 104 at location 128 via welding, adhesives, a clamped or crimped ring or sleeve, or any similar combinations or means. The capture basket 126 may be constructed similarly to the capture basket 102 (e.g., a mesh structure made from memory shape alloy wires, non-memory shape alloy wire, or polymer wires/fibers), and may be a single layer or a double layer mesh structure, as also previously described.

In some examples the movable closure member 121 may be connected near a distal end or region of the inner catheter member 106. For example, the movable closure member 121 may be connected to the inner catheter member 106 at location 122 via welding, adhesives, a clamped or crimped ring or sleeve, or similar combinations or means. The movable closure member 121 may be constructed similarly to previously described capture baskets, such as the capture basket 102. For example, the movable closure member 121 may be a mesh structure made from a shape memory alloy (e.g., Nitinol wires), mesh structure made from non-memory shape alloy wires, or a mesh structure made from polymer wires or fibers. The movable closure member 121 can also be a single layer or a double layer mesh structure, such as also previously described with respect to the capture basket 102. In some alternative examples, the movable closure member 121 may also be composed of a solid material that can self-expand from a radially compressed state to a radially expanded state, such as a solid polymer member or a shape memory scaffold with a membrane positioned over it.

In some examples, the movable closure member 121 may have a diameter in its radially expanded configuration that is similar to a diameter of the distally facing opening 105 of the capture basket 126 in its radially expanded state (e.g., slightly smaller, slightly larger, or about the same size). In FIGS. 6-8, the movable closure member 121 is generally depicted as forming a cone or disc shape, however, the movable closure member 121 can also form other shapes such as, but not limited to, a sphere, cube, a plane with multiple sides, a concave dish, a convex dish, a conical shape, or other similar or different shapes.

In some examples, one or more wires of the one or more wires 110 may also be connected to the inner catheter member 106 and/or the outer catheter member 104 such that the one or more wires 110 may be manually moved, shaped, or expanded by a physician through relative movement (e.g., axial or longitudinal and/or rotational movement) between the outer catheter member 104 and the inner catheter member 106. In one specific example, the one or more wires 110 may be connected to the elongated device body 101 (e.g., inner catheter member 106 and outer catheter member 104) at locations 124 on the inner catheter member 106 and location 128 on the outer catheter member 104 by welding, adhesives, a clamped or crimped ring or sleeve, or any similar combinations or means.

While only a single wire of the one or more wires 110 is shown in FIGS. 6-8, several wires may also be included, such as, but not limited to, 2, 3, 4, 5, 6, or any additional number of individual wires. As seen in FIGS. 6-8, the one or more wires 110 may be positioned at least partially within the cavity of the capture basket 126. If the one or more wires 110 remain at least partially radially expanded as the movable closure member 121 and the inner catheter member 106 are pulled or moved proximally, the helical, looped, or tubular shape formed by the one or more wires 110 may help pull or direct the clot into the cavity of the capture basket 126 and/or further break up the clot into smaller pieces.

In an alternative example, the relative positions of the movable closure member 121 and the capture basket 126 along the elongated device body 101 of the clot retrieval device 120 may be reversed, such that the movable closure member 121 is located proximally of the capture basket 126 rather than distally of the capture basket 126.

FIGS. 6-8 show a progression of how the clot retrieval device 120 may be operated to cut and capture a clot. First, the clot retrieval device 120 may be positioned at a target location within the vasculature of a patient in a variety of different ways, such as including, but not limited to, by following a guidewire (e.g., the inner catheter member 106 may include a guidewire lumen extending between its proximal and distal end), by being advanced through a larger access catheter, or a combination thereof. In some examples, the outer sheath 114 may be used, and may be desirable for, capturing and removing a clot. In other examples, the outer sheath 114 may not be used or needed.

Once the distal region of the clot retrieval device 120 is positioned or located at a target location within the vasculature of a patient (i.e., near a clot), the distal region of the elongated device body 101 clot retrieval device 120 may be advanced through some, or all, of the clot. In one example, this may include advancing the entire distal region of the elongated device body 101 through some, or all, of the clot when the inner catheter member 106 and the outer catheter member 104 are retracted within the outer sheath 114 (or otherwise are in a radially compressed or relatively unexpanded state). The movable closure member 121 may then be expanded to its radially expanded state distally to some, or all, of the clot; and, the capture basket 126 may be expanded to its radially expanded state proximally of some, or all, of the clot (e.g., by distally advancing the inner catheter member 106 and outer catheter member 104, and/or by proximally withdrawing the outer sheath 114). Hence, FIG. 6 illustrates an example of an initially deployed state of the clot retrieval device 120.

The inner catheter member 106 and the outer catheter member 104 may subsequently be moved (e.g., translated axially or longitudinally and/or rotated) relative to each other to achieve a desired amount of radial expansion, or a desired size and shape, for the one or more wires 110. For example, the one or more wires 110 may be formed to (e.g., expanded or moved into) a helical shape that is located around, and is spaced apart from, the inner catheter member 106. Such a helical shape may have a diameter that is about the same size as a diameter of the distally facing opening 105 of the capture basket 126, a diameter of the capture basket 126, and/or the diameter of the target vessel (e.g., adjacent to the vessel wall).

Next, the one or more wires 110 may be moved towards, and at least partially through, the clot so as to enable the one or more relatively sharp edges or surfaces of the one or more wires 110 to cut into and dislodge part, or all, of the clot. This may be accomplished by moving the inner catheter member 106, the outer catheter member 104, or both in unison, such as discussed in detail with respect to FIGS. 1-5 above.

Once all, or a portion of, the clot has been dislodged from the vessel wall, the clot, or the clot pieces, may be moved into the distally facing opening 105 of the capture basket 126. This may be achieved by moving the outer catheter member 104 and capture basket 126 distally, moving the inner catheter member 106 and the movable closure member 121 distally, or both.

As illustrated in FIG. 7, optionally, the one or more wires 110 may be reduced in diameter by the physician at any time after the clot has been cut from the vessel wall, and can, for example, be tightly twisted around the inner catheter member 106, or can remain at a smaller diameter spaced apart from the inner catheter member 106 to help pull the clot into the cavity of the capture basket 126.

As illustrated in FIG. 8, the movable closure member 121 may further be proximally pulled up near, or up against, the distally facing opening 105 of the capture basket 126 to keep or maintain the clot within the cavity, such as if the capture basket 126 is withdrawn into the outer sheath 114. Finally, the clot retrieval device 120 may be withdrawn from the patient, such as by first proximally retracting the inner catheter member 106 and the outer catheter member 104 of the elongated device body 101 at least partially back into the outer sheath 113, if the outer sheath 114 is used, and proximally removing the clot retrieval device 120 from the vasculature of the patient.

Any of the example clot retrieval or removal devices that include an elongated device body 101 including at least two components that move relative to each other, such as, but not limited to, the inner catheter member 106 and outer catheter member 104 in the clot retrieval device 100, the clot retrieval device 120, or any of the additional clot retrieval or removal device discussed below, may include a mechanism at, or near, a proximal end of the clot removal or retrieval device that controls the relative movement of the two components relative to each other. For example, a releasable lock may be included that locks and unlocks (e.g., enables or prevents) the two components (e.g., the inner catheter member 106 and the outer catheter member 104) from moving relative to each other. In another example, an example clot removal or retrieval device can include a handle having at least two separate controls, such as, but not limited to, two thumbwheels that control longitudinal movement of each of the two movable components (e.g., the inner catheter member 106 and the outer catheter member 104), as well as the aforementioned optional releasable lock.

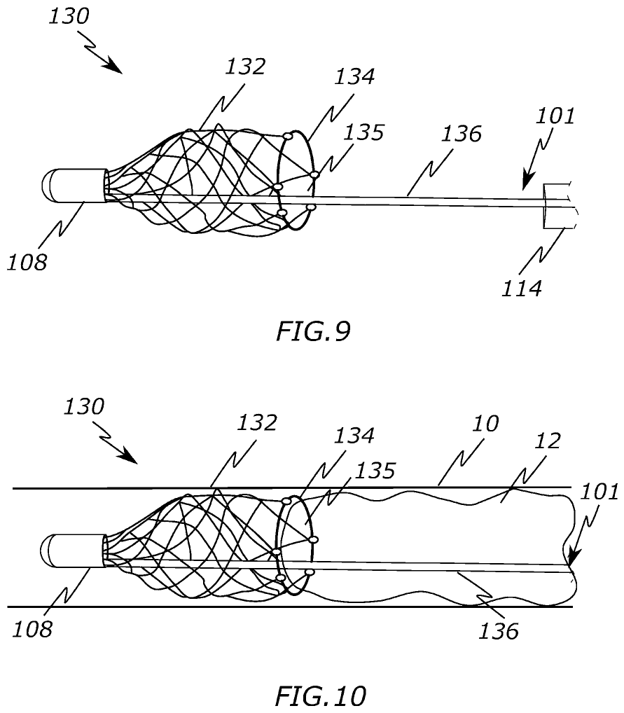

FIGS. 9 and 10 illustrate another example clot retrieval device 130. The clot retrieval device 130 may be similar to the clot retrieval device 100 and the clot retrieval device 120, at least in that the clot retrieval device 130 includes a capture basket 132 and a cutting element, such as, but not limited to, realized in the form of a ribbon or wire 134. However, in contrast to the clot retrieval device 100 and the clot retrieval device 120 discussed with respect to FIGS. 1-8 above, the elongated device body 101 of the clot retrieval device 130 comprises, alternatively to the inner catheter member 106 and the outer catheter member 104, a pusher 136 that may be moved or advanced out of, and retracted back into, the outer sheath 114.

In some examples, a distal region of the pusher 136 (e.g., the elongated device body 101) may include the capture basket 132. As seen in FIGS. 9-10, the capture basket 132 may include a proximally facing opening 135. A distal end of the capture basket 132 may be connected to the pusher 136 at, or near, its distal end, such as via a tip or clamp member 108 (e.g., a metal or polymer tube or cap that is attached to the ends of the mesh tube and to the clot retrieval device 100 via adhesives, welding, crimping, and/or other techniques or means).

The capture basket 132 may be constructed similarly to other capture baskets discussed in this specification, for example, by being formed from a plurality of braided wires or filaments composed of a shape memory alloy (e.g., Nitinol wires), non-shape memory alloy (e.g., stainless steel wires), or polymer filaments or wires (e.g., PET filaments). In some examples, the capture basket 132 may be disposed around the pusher 136, or the pusher may located be outside of the interior cavity of the capture basket 132. The capture basket 132 may be include a single layer, or may include two layers, such as previously discussed with respect to the capture basket 102 or the capture basket 126.

The proximally facing opening 135 defined by the loop shape of the wire 134 may be located at a variety of angles relative to a central or longitudinal axis of the pusher 136. For example, the proximally facing opening 135 defined by the loop shape of the ribbon or wire 134 may be located at an orthogonal or perpendicular angle (e.g., about 90-degrees), or alternatively at a biased angle (e.g., about 45 degrees, about 315-degrees, or otherwise between about 0 degrees and about 89 degrees, or between about 91 degrees and about 180 degrees).

A proximal edge of the capture basket 132 may include, or may be defined by, a ribbon or wire 134 that a relatively sharp edge oriented proximally to enable the capture basket 132 to function as a cutting element. In some examples, the ribbon or wire 134 may include a single wire, or may include be a plurality of individual wires or wire segments, that may extend around the proximally facing opening 135 of the inner cavity or interior formed by the capture basket 132. The ribbon or wire 134 may expand to a radial diameter that is similar to the radial diameter of a target vessel such that the helical, loop, or tubular shape formed by the ribbon or wire 134 may help separate a clot from the target vessel wall. In some examples, the ribbon or wire 134 may be retained by loops or other features of the capture basket 132 located radially around the proximally facing opening 135.

As illustrated in FIG. 10, in the operation of some examples of the clot retrieval device 130, a distal end of the elongated device body 101 (e.g., the pusher 136 of the clot retrieval device 130 may first be advanced partially, or fully, beyond a clot 12 located in a target vessel 10. The outer sheath 114 may then be proximally withdrawn to allow the capture basket 132 to radially expand from a radially compressed state to a radially expanded state, such as seen in FIG. 10. The pusher 136 may then be moved proximally so that the ribbon or wire 134 contacts and/or cuts (or pulls) the clot 12 away from the vessel wall 10, and, in turn, allowing at least a portion of the clot 12 to move into the cavity of the capture basket 132. The capture basket 132 can subsequently be partially, or fully, withdrawn into the outer sheath 114. Finally, the clot retrieval device 130 may be removed from the patient. As with all the example clot retrieval or removal devices described in this specification, multiple cutting passes through the clot may be possible or desirable to entirely retrieve or dissect the clot, such as during the removal of relatively long clots.

While the capture basket 102, the capture basket 126, the capture basket 132, or the capture element 192, the cutting and capture element 212, the cutting and capture element 232, or any other clot capture element or structure discussed in this specification above or below are generally described as being oriented in specific proximal or distal orientations, it is to be appreciated that these orientations or directions may be reversed. Further, any clot capture element or structure discussed in this specification above or below may be connected to different movable components to those described in this specification so as to, for example, capture a clot from a different direction relative to the directions described in this specification.

As previously discussed, the various cutting elements discussed in this specification, such as including the one or more wires 110, the ribbon or wire 134, or the capture basket 132, may have or form a variety of different cross-sections, cross-sectional shapes, or other shapes. It is to be appreciated that such cross-sections, cross-sectional shapes, or other shapes of these cutting elements, and any other cutting element described below, may include at least one relatively sharp location or surface (e.g., one or more cutting edges) that may help cut into a clot and/or move the clot to a desired location along the vessel. Various non-limiting examples of such cross-sections and cross-sectional shapes of a cutting element are shown in FIGS. 11-17.

FIG. 11 illustrates a cutting element 140 having a rectangular cross-section. This may create four edges along at least a portion of the length of the cutting element 140. In one example, this cross-sectional shape may be created by laser cutting a thin tube of material (e.g., metal).

FIG. 12 illustrates a cutting element 142 having a generally rectangular cross-section and a pointed or triangular end. The pointed or triangular end may be oriented proximally or distally, depending on the position and orientation of a clot capture basket or other clot capture element.

FIG. 13 illustrates a cutting element 144 having a generally rectangular or hexagonal cross-section and two pointed or triangular ends. At least one of the pointed or triangular ends may be orientated proximally or distally, depending on the position and orientation of a clot capture basket or clot capture element.

FIG. 14 illustrates a cutting element 146 having a generally rectangular cross-sectional shape that is bent or curved, or that might otherwise be considered concave or convex.

FIG. 15 illustrates a cutting element 148 with a generally rectangular cross-sectional shape with one or more pointed or triangular portions along a side (i.e., a longer, flatter side vs. shorter ends as in the cutting elements 142 and the cutting element 144).

FIG. 16. illustrates a cutting element 150 with a generally circular cross-sectional shape with one or more pointed or triangular portions.

FIG. 17 illustrates a cutting element 152 with a generally star cross-sectional shape with a plurality of pointed or triangular portions.

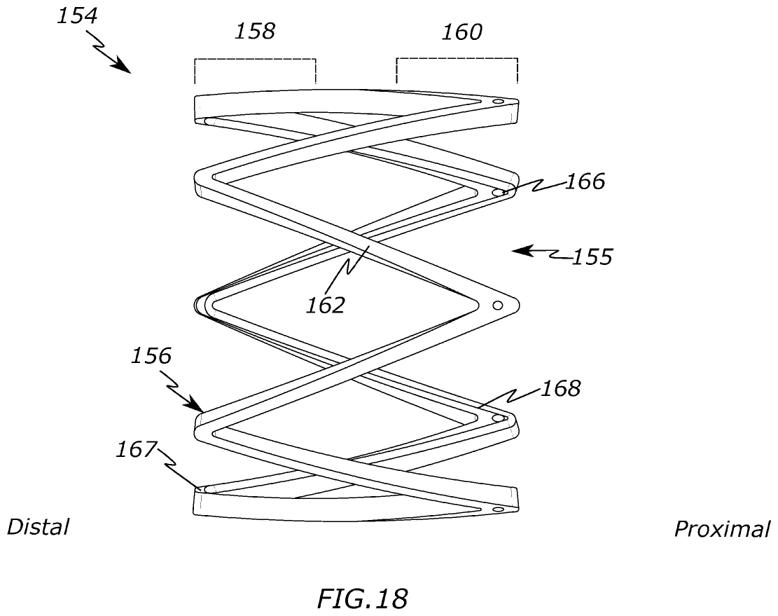

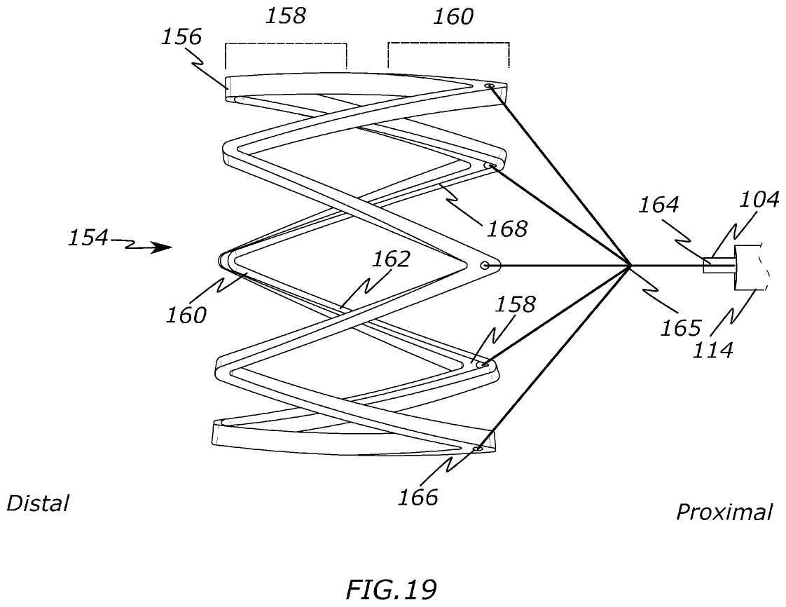

FIGS. 18-19 illustrate an example of a cutting element 154. Also shown in FIGS. 18-19 are orientation indicators “Proximal” and “Distal.” FIGS. 18-19 are discussed below concurrently. The cutting element 154 may form a full or complete loop (e.g., a tubular or stent-like structure), which may be defined and supported by a plurality of struts 156. In various examples, the plurality of struts 156 may include, but is not limited to, 4, 6, 8, 10, 12, 14, 16, 18, 20, or other numbers of individual struts.

In some examples, such as shown in FIGS. 18-19, each strut of the plurality of struts 156 may be connected to another strut of the plurality of struts 156 to form a V-shape. In other examples, each of the plurality of struts 156 may be connected to another other to form a zigzag shape or pattern, an alternating wave shape or pattern, or other arrangements or patterns. In some examples, one or more of the plurality of struts 156 may be tapered, scalloped, angled, or can otherwise define one or more varying dimensions between a proximal region 158 and a distal region 160 of each strut.

For example, a width of at least one of the plurality of struts 156 measured at or near the proximal region 158 of each strut, may be wider or greater than a width of the at least one strut of the plurality of struts 156 measured at the distal region 160 of each strut. In another example, a width of at least one of the plurality of struts 156 measured at or near the proximal region 158 of each strut, may be narrower or lesser than a width of the at least one strut of the plurality of struts 156 measured at the distal region 160 of each strut. In another example, a width of at least one of the plurality of struts 156 measured at or near the proximal region 158 and the distal region 160 of each strut, may be wider or greater, or alternatively, narrower or lesser, than a width of the at least one strut of the plurality of struts 156 measured at a middle region 162 of each strut extending therebetween.

The plurality of struts 156 may be composed of, for example, but not limited to, a shape memory alloy (e.g., Nitinol), a non-shape memory alloy (e.g., stainless steel), or a polymer (e.g., polyethylene terephthalate “PET”). In the case of a shape memory alloy, the plurality of struts 156 may be shape-set to expand the loop or tubular shape of the cutting element 154 radially outward, such from a compressed configuration to a radially expanded configuration, such as shown in FIG. 18-19. In the radially expanded configuration, the cutting element 154 can define a diameter that is either smaller than, or that is similar to, the diameter of a target vessel and/or a diameter of any capture element discussed in this disclosure.

As illustrated in FIG. 19, the cutting element 154 may be controllable in its radial size relative to the vasculature of a patient. This may generally be achieved by connecting the plurality of struts 156 to movable components (e.g., the outer catheter member 104 and the inner catheter member 106) of the elongated device body 101 via a wire 164. In some such examples, a proximal end of the wire 164 may be connected to the outer catheter member 104 via adhesives, welding, clamping (e.g., via one or more clamps) and/or other similar means or techniques. In one specific example, the proximal end of the wire 164 may be connected to the outer catheter member 104 at location 112, and a distal perimeter (e.g., one or more struts defining a defining a distal-most surface of the cutting element 154) may be connected to the capture basket 102, such as in a position abutting the proximally facing edge 102A. In such an example, the cutting element 154 may be used alternatively, or in place of, the one or more wires 110.

The distal end 165 of the wire 164 may be connected to the cutting element 154 in a variety of different ways. For example, such as shown in FIG. 19, the cutting element 154 may include a plurality of apertures 166 each sized and shaped to enable a length, or an individual, strand of the wire 164 to pass therethrough. In such an example, each of the plurality of apertures 166 may be defined at, or near, proximal apexes 167 (e.g., locations formed by the intersection of two struts). In another example, a length or strand of the wire 164 may be wrapped, or tied around, two or more of the proximal apexes 167. In another example, one or more distal ends of the wire 164 may be welded adhered, clamped, or otherwise directly affixed to the proximal region 158 of two or more struts of the plurality of struts 156.

The cutting element 154 may define a plurality of cutting surfaces 168. For example, at least two struts of the plurality of struts 156 may be scalloped, angled, tapered, or otherwise shaped to form a cutting surface that is oriented inwardly and proximally (e.g., configured to cut via proximal motion of the cutting element 154 relative to a vessel wall). The plurality of cutting surfaces 168 may be dictated by the cross-sectional shape of each strut of the plurality of struts 156. For example, each strut of the plurality of struts 156 may define a cross-section, or cross-sectional shape, that is similar to any of the cross-sections, or cross-sectional shapes, having one or more cutting edges or surfaces described above with regard to any of FIGS. 11-17.

The cutting element 154 may be located at a variety of angles relative to a central or longitudinal axis of the elongated device body 101. For example, a proximal opening 155 defined by the tubular shape of the cutting element 154 may be located at an orthogonal or perpendicular angle (e.g., about 90-degrees), or at a biased angle (e.g., about 45 degrees, about 315-degrees, or otherwise between about 0 degrees and about 89 degrees, or between about 91 degrees and about 180 degrees, relative to the inner catheter member 106 or the outer catheter member 104.

In some examples, the cutting element 154 may be configured to create additional radial force during expansion, or otherwise have an increased radial stiffness, such as to help keep the cutting element 154 aligned with a central axis of a vessel wall during cutting of a clot. For example, alternatively to the single row (or column) arrangement of the plurality of struts 156 shown in FIGS. 18-19, the cutting element 154 may include two, three, four, five, six, or other additional rows (or columns) of struts connected to, and extending laterally distally from, the plurality of struts 156 shown in FIGS. 18-19. In one such example, each distal region 160 of the plurality of struts 156 may be connected to an adjacent area or portion of the proximal region 158 of a strut of an adjacent row of struts to thereby form a multi-row arrangement. In a further example, the wire 164 may further be constructed to impart an expansion force to the plurality of struts 156, such as to help increase the radial expansion force at which the cutting element 154 expands from the radially retracted configuration to the radially expanded configuration.

The cutting element 154 may be manufactured using various manufacturing or production techniques. For example, the cutting element 154, including the plurality of struts 156 and the plurality of cutting surfaces 168 thereof, may be laser cut, three-dimensionally printed, metallically molded, or the plurality of struts 156 may be welded together. In one specific example, the plurality of cutting surfaces 168 may be mechanically ground, laser cut, or alternatively chemically etched, into the cutting element 154.

In the operation of some examples of a clot retrieval or removal device in accordance with the present disclosure, the cutting element 154 may be used to cut and help capture a clot. First, a clot retrieval or removal device that includes the cutting element 154, such as in place of the one or more wires 110, may be positioned at a target location within vasculature of a patient in a variety of different ways, such as, but not limited to, those discussed above with regard to FIGS. 1-10. Once the clot retrieval or removal device is positioned at the target location (i.e., near a clot), a distal region or portion of the clot retrieval or removal device may be advanced through some, or all, of the clot.

In some examples, if the outer sheath 114 is used, both the inner catheter member 106 (not shown in FIGS. 18-19) and the outer catheter member 104 can then be advanced distally out of the outer sheath 114 to, in turn, allow the cutting element 154 to radially expand from its radially compressed configuration to its radially expanded configuration, such as seen in FIGS. 18-19. The inner catheter member 106 and the outer catheter member 104 may also be moved (e.g., translated axially longitudinally and/or rotated) relative to each other to achieve a desired degree of radial expansion, or a desired size and shape, for the cutting element 154.

Next, the cutting element 154 may be moved proximally toward the clot so that the cutting surfaces 168 can cut into the clot, such as by moving the inner catheter member 106 and the outer catheter member 104 proximally. As the cutting element 154 is moved proximally into the clot, the inner catheter member 106 may be moved relative to the outer catheter member 104 to build or generate tension within the wire 164, and, in turn, cause the cutting element 154 to radially compress or otherwise shrink in diameter. As may be appreciated, this can cause the plurality of cutting surfaces 168 to engage and cut the clot in a scissor-style motion or action, which may be advantageous in help to shear one or more pieces from the clot or cut the entire clot from a vessel wall.

In some examples, the cutting element 154 may subsequently be further proximally translated until the clot, or the sheared pieces thereof, enter the interior or inner cavity of the capture basket 102 (not shown in FIGS. 18-19) through the proximally facing edge 102A (not shown in FIGS. 18-19) abutting the cutting element 154. The clot, or the sheared pieces thereof, may also be retained within the capture basket 102. For example, the inner catheter member 106 may again be moved, or maintained in a position, relative to the outer catheter member 104 to build or generate tension within the wire 164, and, in turn, cause the cutting element 154 and the proximally facing edge 102A connected thereto to radially contract, thus closing the capture basket 102 in a process similar as shown in FIGS. 2-4.

Finally, the clot retrieval or removal device may be removed from the patient, such as by first proximally retracting the elongated device body 101 at least partially into the outer sheath 114, and then proximally withdrawing the outer sheath 114 from the vasculature of the patient.

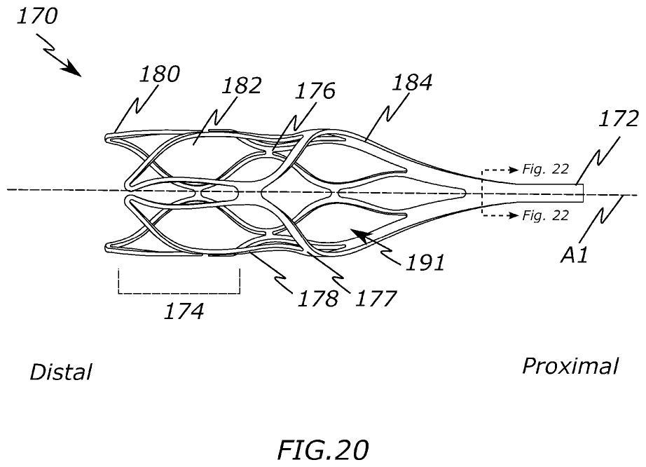

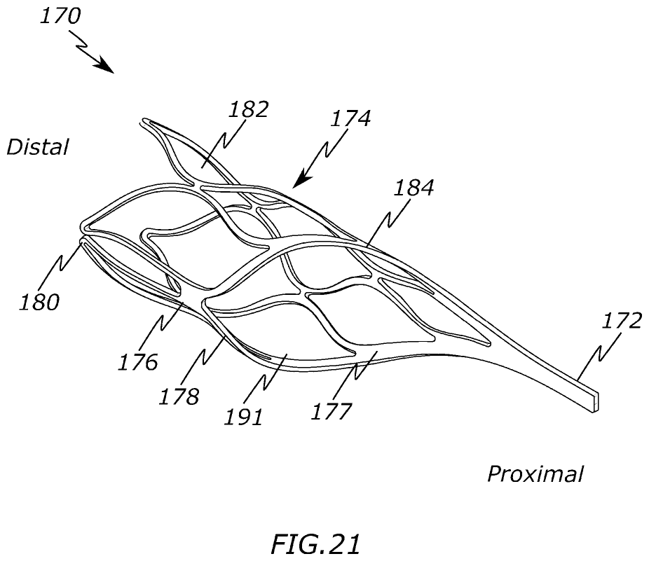

FIGS. 20-21 illustrate another example of a cutting element 170. FIGS. 20-21 are discussed below concurrently. In particular, FIG. 20 illustrates a top view of the cutting element 170 in a radially expanded configuration and FIG. 21 illustrates a side view of the cutting element of FIG. 20 in the radially expanded configuration. Also shown, FIGS. 20-21 illustrate orientation indicators “Proximal” and “Distal.” A longitudinal axis A1 (FIG. 20) may be generally representative of an axis extending centrally through the elongated device body 101 or the cutting element 170. The cutting element 170 may include a proximal portion 172 and a distal portion 174. The proximal portion 172 and the distal portion 174 may generally be opposite areas or sections of the cutting element 170. For example, the proximal portion 172 may define, but not limited to, about 5, 10, 15, or 20 percent of the overall longitudinal length of the cutting element 170, as measured relative to the longitudinal axis A1, and the distal portion 174 may define, but not limited to, about 95, 90, 85, or 80 percent of the overall longitudinal length of the cutting element 170, as measured relative to the longitudinal axis A1, respectively.

In some examples, the proximal portion 172 may generally form a curved and relatively thin cross-sectional profile, such as adapted to correspond to an outer surface of the elongated device body 101 (not shown in FIGS. 20-21). In one example, the proximal portion 172 may be an elongated strut or body extending proximally from the distal portion 174 along the longitudinal axis A1. In some examples, the distal portion 174 may include a plurality of struts 176 and a loop 177. In some examples, such as shown in FIGS. 20-21, the plurality of struts 176 may be representative of a plurality of elongated members connected to one another in various arrangements to form a semi-annular structure (e.g., a partial tube) or an annular structure (e.g., a stent-like tube). Each strut of the plurality of struts 176 may be connected to another strut of the plurality of struts to define, for example, but not limited to, a repeating zigzag arrangement or pattern, a repeating alternating wave arrangement or pattern, a repeating V-shape or pattern.

In some examples, such as shown in FIGS. 20-21, each of the plurality of struts 176 may define similar or identical dimensions between opposite proximal portions 178 and distal portions 180 thereof. In other examples, each strut, or one or more struts of, the plurality of struts 176 may be tapered, scalloped, angled, or may otherwise define one or more varying dimensions between the opposite proximal portions 178 and the distal portions 180 thereof. Each of the plurality of struts 176 may also be sized and shaped, and connected to one another, to form a plurality of cells 182 adapted to enable blood or other fluids to flow therethrough. The size and shape of each of the plurality of cells 182 may depend on the size, shape, and arrangement of each of the plurality of struts 176 relative to one another, as well as a desired porosity of the cutting element 170.

The loop 177 may be a proximal-most, solid portion of the distal portion 174 from which each of the proximal portions 178 of the plurality of struts 176 may distally extend. In some examples, such as shown in FIGS. 20-21, the loop 177 may be a complete or closed loop (e.g., an annular shape or ring extending 360 degrees about the longitudinal axis A1). In other examples, the loop 177 may be a partial or incomplete loop (e.g., a curved shape extending less than 360 degrees about the longitudinal axis A1). The loop 177 may define one or more cutting surfaces 184. The one or more cutting surfaces 184 may generally represent one or more relatively sharp, scalloped, angled, tapered, or otherwise deliberately shaped, surfaces of the loop 177. Each of the one or more cutting surfaces 184 can be oriented proximally (e.g., configured to cut a clot via proximal translation of the cutting element 170 relative to a vessel wall).

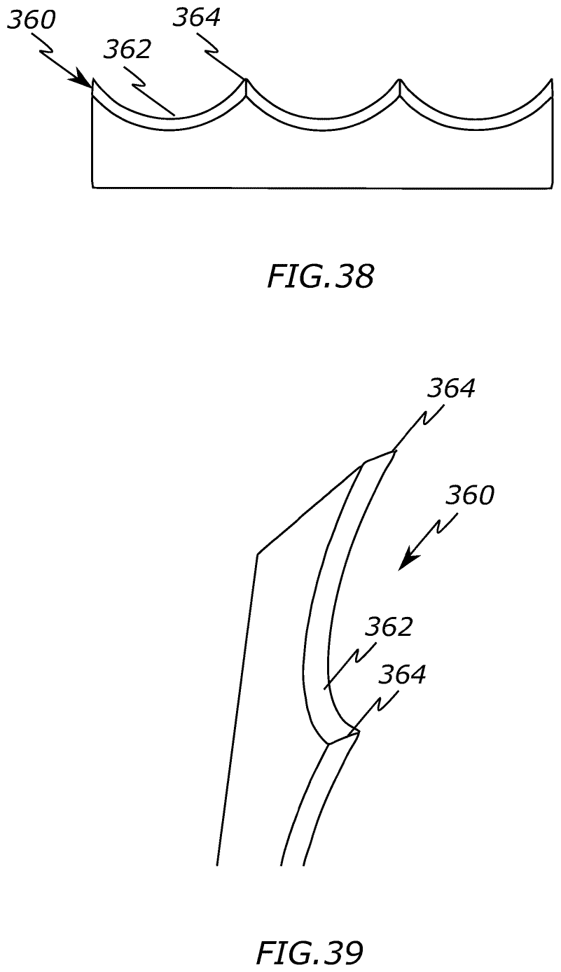

In some examples, the one or more cutting surfaces 184 may be defined by the cross-sectional shape of the loop 177. For example, the loop 177 may define a cross-section shape that is similar to any of the cross-sections, or the cross-sectional shapes, each having one or more cutting edges or surfaces shown FIGS. 11-17. In one example, the one or more cutting surfaces 184 may be a single chamfered surface defined at an acute angle relative to the longitudinal axis A1. In some examples, the surface area of one or more cutting surfaces 184 may be smooth or polished, or alternatively, the surface area of the one or more cutting surfaces 184 can be serrated, such as by including a variety of different cutting projections or protrusions, or by including any of the serrated cutting elements or edges discussed with reference to FIGS. 38-48 below.

The one or more cutting surfaces 184 can also be defined in various positions or orientations about the loop 177. For example, the one or more cutting surfaces 184 may be defined along an inner surface of the loop 177, such as shown in FIG. 20, along an outer surface of the loop 177, such as shown in FIG. 21, at one or more locations between the outer surface and the inner surface of the loop 177, or otherwise along one or more side surfaces of the loop 177 or the plurality of struts 176 of the distal portion 174.

The loop 177, and the one or more cutting surfaces 184 defined thereby, may be positioned at various angles with respect to the longitudinal axis A1. For example, a proximal opening 191 defined by the loop 177 may extend orthogonally (e.g., about 90-degrees) to the longitudinal axis A1. Alternatively, the proximal opening 191 defined by the loop 177 may extend at a biased angle relative to the longitudinal axis A1 (e.g., at about 45 degrees, about 315-degrees, between about 0 degrees and about 89 degrees, or between about 91 degrees and about 180 degrees.

Moreover, the loop 177 and/or the one or more cutting surfaces 184 may be located at a single angle with respect to the longitudinal axis A1 or may alternatively be located at multiple angles concurrently with respect to the longitudinal axis A1. For example, the one or more cutting surfaces 184, or a proximal-most face or surface of the loop 177, may be a planar surface which extends along only a single tangent with respect to the longitudinal axis A1 or may be a curved surface which extends along multiple tangents with respect to the longitudinal axis A1.

The cutting element 170 may be composed of, for example, but not limited to, a shape memory alloy (e.g., Nitinol), a non-shape memory alloy (e.g., stainless steel), or a polymer (e.g., polyethylene terephthalate “PET”). In the case of a shape memory alloy, the distal portion 174 and the loop 177 may be shape-set to expand from a radially compressed configuration to a radially expanded configuration when unconstrained. In the radially expanded configuration, such as of the example cutting element 170 shown in FIGS. 20-21, the distal portion 174 and the loop 177 of the cutting element 170 can define a diameter that is either smaller, or that is similar to, a diameter of a target vessel and/or a diameter of any capture element discussed in this disclosure.

The cutting element 170 may be manufactured using various manufacturing techniques. For example, the cutting element 170, including the one or more cutting surfaces 184, may be laser-cut, three-dimensionally printed, or metallically molded. In one example, the one or more cutting surfaces 184 may be mechanically ground into the cutting element 170, or alternatively, chemical etched in the cutting element 170. In some examples, the clot removal devices 190, 210, and 230 described below may each utilize a cutting element that is similar to the cutting element 170.

The cutting element 170 may define or otherwise form various dimensions. For example, some, or all, portions or segments of the cutting element 170, such as including any of the proximal portion 172, the distal portion 174, the loop 177, each of the plurality of struts 176, or each of the one or more cutting surfaces 184, may have a wall thickness measuring between, but not limited to, about 0.1 millimeter and about 1 millimeter. In various examples, the loop 177 may define an outer diameter measuring between, but not limited to, about 1 millimeter and about 6 millimeters, depending on which configuration the cutting element 170 is in.

For example, the loop 177 may define an outer diameter, in a radially expanded configuration, measuring between, but not limited to, about 4 millimeters and about 7 millimeters. In one example, the loop 177 may define an outer diameter, in a radially expanded configuration, measuring about 5.4 millimeters. In some examples, the loop 177 may define an outer diameter, in a radially compressed configuration, measuring between, but not limited to, about 1 millimeter and about 3 millimeters. In one example, the loop 177 may define an outer diameter, in a radially compressed configuration, measuring about 1.37 millimeters.

In various examples, the cutting element 170 may have a longitudinal length, such as defined axially between the proximal portion 172 and the distal portion 174, measuring between, but not limited to, about 15 millimeters and about 25 millimeters. In one example, the longitudinal length of the cutting element 170 may measure about 20 millimeters. Further, the proximal portion 172 may extend along, or otherwise may comprise, various longitudinal distances of the total longitudinal length of the cutting element 170, such as measured before a proximal most-point of the one or more cutting surfaces 184. For example, a longitudinal distance from a proximal end point of the proximal portion 172 to a proximal-most point of the one or more cutting surfaces 184, or of the distal portion 174, may measure between, but not limited to, about 1 millimeter and about 5 millimeters. In one example, such when the overall longitudinal length of the cutting element 170 measures about 20 millimeters, such a longitudinal distance may measure about 2.3 millimeters.

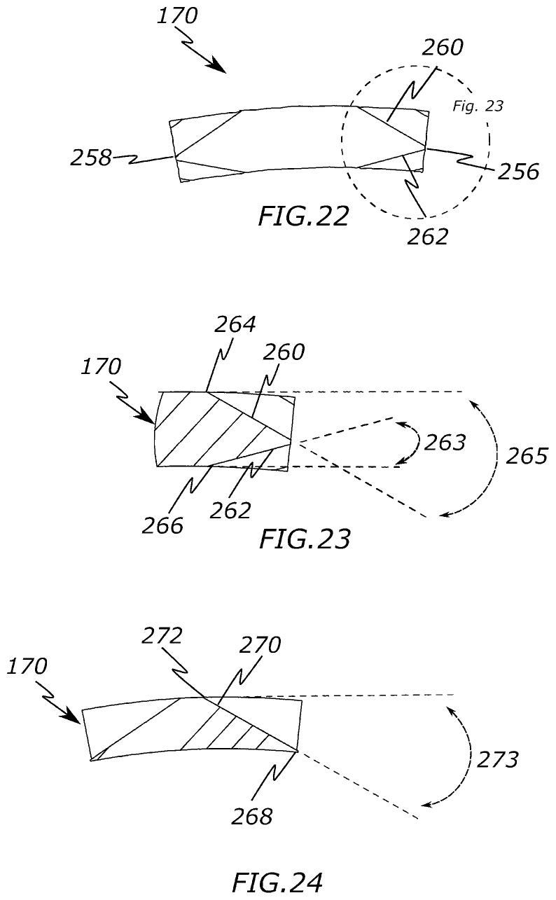

FIG. 22 illustrates a cross-section of the cutting element 170 along the cross sectional lines shown in FIG. 20, while FIG. 23 illustrates an enlarged view of the cross section shown in FIG. 22. As previously discussed above, the one or more cutting surfaces 184 (FIGS. 20-21) of the cutting element 170 may include various numbers of individual cutting surfaces, which may be adapted to and extend at various angles with respect to the longitudinal axis A1 (FIGS. 20-21) or other reference points. In some examples, such as shown in FIGS. 22-23, the one or more cutting surfaces 184 may include a first cutting surface 256 and a second cutting surface 258. The first cutting surface 256 and the second cutting surface 258 may generally be opposite innermost and outermost surfaces of the cutting element 170, such as defined along a length or section of any of the distal portion 174 (FIGS. 20-21), the loop 177 (FIGS. 20-21), the proximal portion 172 (FIGS. 20-21), or a combination thereof. In other examples, only the first cutting surface 256 or the second cutting surface 258 may be included.

Referring to FIG. 23, for the sake of brevity, the following discussion is directed to the first cutting surface 256. However, it is to be appreciated that the second cutting surface 258 may also include the same, similar, or different features or attributes. In some examples, the first cutting surface 256 may be defined between an upper surface 260 and a lower surface 262 of the cutting element 170. The upper surface 260 may form an angle 263 with respect to a point or tangent 264 along an exterior of the cutting element 170. The lower surface 262 may form an angle 265 with respect to a point or tangent 266 along an interior of the cutting element 170. In some examples, the angle 263 and the angle 265 may be similar or identical angles, such as, but not limited to, angles measuring between about 10 degrees and about 35 degrees.

In other examples, the angle 263 and the angle 265 may be different angles. In some such examples, the angle 263 may be an angle measuring between, but not limited to, about 15 degrees and about 45 degrees, and the angle 265 may be an angle measuring between, but not limited to, about 5 degrees and about 30 degrees. In one example, the angle 263 may be angle measuring about 30 degrees and the angle 265 may be an angle measuring about 15 degrees.

FIG. 24 illustrates an alternate cross-sectional shape of the cutting element 170 along similar cross sectional lines as shown in FIG. 22. As previously discussed above, the one or more cutting surfaces 184 (FIGS. 20-21) of the cutting element 170 may include various numbers of individual cutting surfaces, which may be adapted extend at various angles with respect to the longitudinal axis A1 (FIG. 20) or other reference points. In some examples, the one or more cutting surfaces 184 of the cutting element 170 may include a single or an individual cutting surface, such as shown in FIG. 24. In such examples, the cutting surface 268 may form an angle 273 between a planar surface 270 and a point 272 along an outer surface of the cutting element 170.

In some such examples, the angle 273 may be an angle measuring between about, but not limited to, about 15 degrees and about 45 degrees. In one example, the angle 273 may be angle measuring about 30 degrees. In some examples, the one or more cutting surfaces 184 may form two cutting surfaces, such as cutting surfaces similar to the first cutting surface 256 and the second cutting surface 258 of the cutting element 170 described with reference to FIGS. 22-23 above. In further examples, the one or more cutting surfaces 184 may include other numbers, or other shapes, of cutting surfaces, such as previously described above with respect to FIGS. 20-21 or FIGS. 11-17.

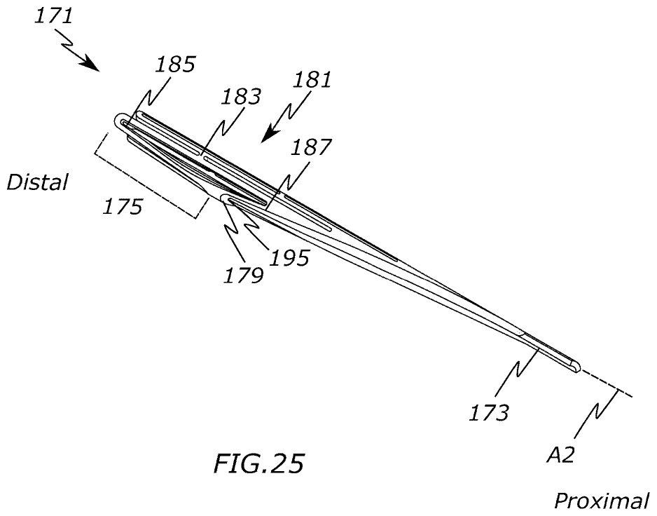

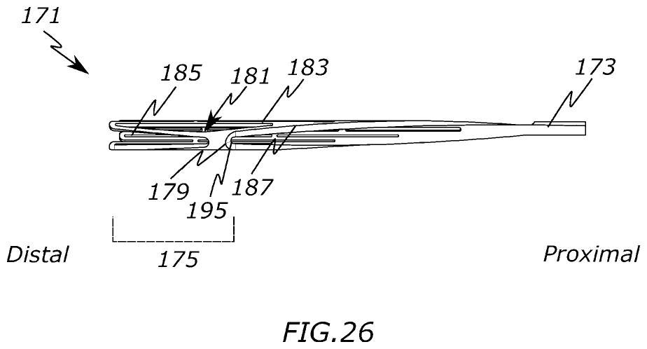

FIGS. 25-27 illustrate another example of a cutting element 171. Also shown in FIGS. 25-27 are orientation indicators “Proximal” and “Distal.” FIGS. 25-27 are discussed below concurrently. A longitudinal axis A2 (FIG. 25), may be generally representative of an axis extending centrally through the elongated device body 101 or the cutting element 171. The cutting element 171 may include a proximal portion 173 and a distal portion 175. The proximal portion 173 and the distal portion 175 may generally be opposite areas or sections of the cutting element 171. For example, the proximal portion 173 may define, but not limited to, about 5, 10, 15, or 20 percent of the overall longitudinal length of the cutting element 171, as measured relative to the longitudinal axis A2, and the distal portion 175 may define, but not limited to, about 95, 90, 85, or 80 percent of the overall longitudinal length of the cutting element 171, as measured relative to the longitudinal axis A2, respectively.

In some examples, the proximal portion 173 may generally form a curved and relatively thin cross-sectional profile, such as adapted to correspond to an outer surface of the elongated device body 101. In one example, the proximal portion 173 may be an elongated strut or body extending proximally from the distal portion 175 parallel to the longitudinal axis A2. The distal portion 175 may include a loop 179. In some examples, such as shown in FIGS. 25-27, the loop 179 may be a complete or closed loop (e.g., an annular shape or ring extending 360 degrees about the longitudinal axis A1). In other examples, the loop 179 may be a partial or incomplete loop (e.g., a curved shape extending less than 360 degrees about the longitudinal axis A2). The loop 177 may generally be a proximal-most, solid portion of the distal portion 174. The cutting element 171 may also include a porous area 181, such as comprised by a plurality of solid portions 183 and a plurality of cells 185 defined therebetween. The plurality of solid portions 183 may at least partially form and support the loop 179, as well as any of other portion of the distal portion 175, such as in a manner similar to the plurality of struts 176 described with regard to FIGS. 20-21 above.

The porous area 181 may be adapted to enable blood or other fluids to flow or pass therethrough. For example, each of the plurality of cells 185 thereof may be selectively sized and shaped based on a desired porosity of the cutting element 171. Each of the plurality of cells 185 may further define similar or identical dimensions, and may form various shapes, such as, but not limited to, a diamond-like shape, an oval shape, a circular shape, rectangular shape, or a wide variety of other shapes.

The cutting element 171 may define one or more cutting surfaces 187. The one or more cutting surfaces 187 may generally represent one or more relatively sharp, scalloped, angled, tapered, or otherwise deliberately shaped, surfaces extending proximally from the loop 179. Each of the one or more cutting surfaces 187 man be oriented proximally (e.g., configured to cut a clot via proximal translation of the cutting element 171 relative to a vessel wall). In some examples, the one or more cutting surfaces 187 may be defined by the cross-sectional shape of the loop 179.

For example, the loop 179 or other portions of the cutting element 171 may define a cross-sectional shape that is similar to any of the cross-sections, or the cross-sectional shapes, each having one or more cutting edges or surfaces shown FIGS. 11-17. In one example, the one or more cutting surfaces 187 may be a single chamfered surface defined at an acute angle relative to the longitudinal axis A2. In some examples, the surface area of one or more cutting surfaces 187 may be smooth or polished, or alternatively, the surface area of the one or more cutting surface 187 can be serrated, such as by including a variety of different cutting projections or protrusions, or by including any of the serrated cutting elements or edges discussed with reference to FIGS. 38-48 below.

The one or more cutting surfaces 187 may also be defined in various positions or orientations about the loop 179. For example, the one or more cutting surfaces 187 may be defined along an inner surface of the loop 179, an outer surface of the loop 179, at one or more locations between the outer surface and the inner surface of the loop 179, or otherwise along one or more side surfaces of the loop 177 or the plurality of struts 176 of the distal portion 174. The loop 179, and the one or more cutting surfaces 187 defined thereby, may be positioned at various angles with respect to the longitudinal axis A2. For example, a proximal opening 195 defined by the loop 179 may extend orthogonally (e.g., about 90-degrees) to the longitudinal axis A2. Alternatively, the proximal opening 195 defined by the loop 179 may extend at a biased angle relative to the longitudinal axis A1 (e.g., at about 45 degrees, about 315-degrees, between about 0 degrees and about 89 degrees, or between about 91 degrees and about 180 degrees.

Moreover, the loop 179 and/or the one or more cutting surfaces 187 may be located at a single angle with respect to the longitudinal axis A2 or may alternatively be located at multiple angles concurrently with respect to the longitudinal axis A2. For example, the one or more cutting surfaces 187, or a proximal-most face or surface of the loop 179, may be a planar surface which extends along only a single tangent with respect to the longitudinal axis A2 or may be a curved surface which extends along multiple tangents with respect to the longitudinal axis A2.

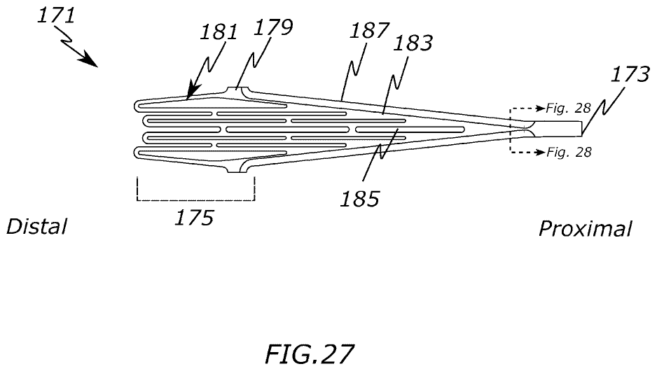

The cutting element 171 may be composed of, for example, but not limited to, a shape memory alloy (e.g., Nitinol), a non-shape memory alloy (e.g., stainless steel), or a polymer (e.g., polyethylene terephthalate “PET”). In the case of a shape memory alloy, the distal portion 175 and the loop 179 may be shape-set to expand from a radially compressed configuration to a radially expanded configuration when unconstrained. In the radially expanded configuration, such as of the example cutting element 171 shown in FIG. 27, the distal portion 175 and the loop 179 of the cutting element 171 can define a diameter that is either smaller, or that is similar to, a diameter of a target vessel and/or a diameter of any capture element discussed in this disclosure.

The cutting element 171 may be manufactured using various manufacturing techniques. For example, the cutting element 171, including the one or more cutting surfaces 187, may be laser-cut, three-dimensionally printed, or metallically molded. In one example, the one or more cutting surfaces 187 may be mechanically ground into the cutting element 171, or alternatively, chemical etched in the cutting element 171. In some examples, the clot removal devices 190, 210, and 230 described below may each utilize a cutting element that is similar to the cutting element 171.

The cutting element 171 may define or otherwise form various dimensions. For example, some, or all, portions or segments of the cutting element 171, such as including any of the proximal portion 173, the distal portion 175, the loop 179, each of the plurality of solid portions 183, or each of the one or more cutting surfaces 187, may have a wall thickness of between, but not limited to, about 0.1 millimeters and about 1 millimeter. In various examples, the loop 179 may define an outer diameter measuring between, but not limited to, about 1 millimeter and about 6 millimeters, depending on which configuration the cutting element 171 is in.

For example, the loop 179 may define an outer diameter, in a radially expanded configuration, measuring between, but not limited to, about 4 millimeters and about 7 millimeters. In one example, the loop 179 may define an outer diameter, in radially expanded configuration, measuring about 5.4 millimeters. In some examples, the loop 179 may define an outer diameter, in a radially compressed configuration, measuring between about 1 millimeter and about 3 millimeters. In one example, the loop 179 may define an outer diameter, in a radially compressed configuration, measuring about 1.37 millimeters.

In various examples, the cutting element 171 may have a longitudinal length, such as defined axially between the proximal portion 173 and the distal portion 175, of between, but not limited to, about 15 millimeters and about 25 millimeters. In one example, the longitudinal length of the cutting element 171 may measure about 22.6 millimeters. Further, the proximal portion 172 may extend along, or otherwise may comprise, various longitudinal distances of the total longitudinal length of the cutting element 171, such as measured before a proximal most-point of the one or more cutting surfaces 187. For example, a longitudinal distance from a proximal end point of the proximal portion 173 to a proximal-most point of the one or more cutting surfaces 187, or of the distal portion 175, may measure between, but not limited to, about 1 millimeter and about 5 millimeters. In one example, such when the overall longitudinal length of the cutting element 170 measures about 22.6 millimeters, such a longitudinal distance may measure about 2.8 millimeters.

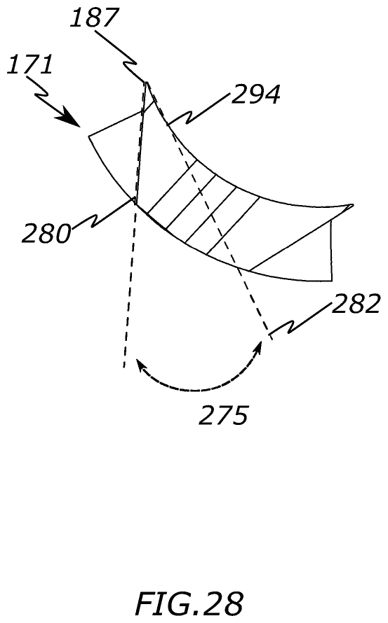

FIG. 28 illustrates a cross-section of the cutting element 171 discussed above with reference to FIGS. 25-27. As previously discussed above, the one or more cutting surfaces 187 (FIGS. 25-27) of the cutting element 171 may include various numbers of individual cutting surfaces, which may be adapted extend at various angles with respect to the longitudinal axis A2 (FIG. 25) or other reference points. In some examples, the one or more cutting surfaces 187 of the cutting element 171 may include a single or an individual cutting surface, such as shown in FIG. 28. In such examples, the one or more cutting surfaces 187 may form an angle 275 between a first tangent 280, and a second tangent 282 intersecting a point 294 along an exterior of the cutting element 171.

In various examples, the angle 275 may be an angle measuring between, but not limited to, about 10 degrees and about 35 degrees. In one example, the angle 275 may be angle measuring about 30 degrees. In some examples, the one or more cutting surfaces 187 may form two cutting surfaces, such as cutting surfaces similar to the first cutting surface 256 and the second cutting surface 258 of the cutting element 170 described with reference to FIGS. 22-23 above. In further examples, the one or more cutting surfaces 187 may include other numbers, or other shapes, of cutting surfaces, such as previously described above with respect to FIGS. 20-21 or FIGS. 11-17.

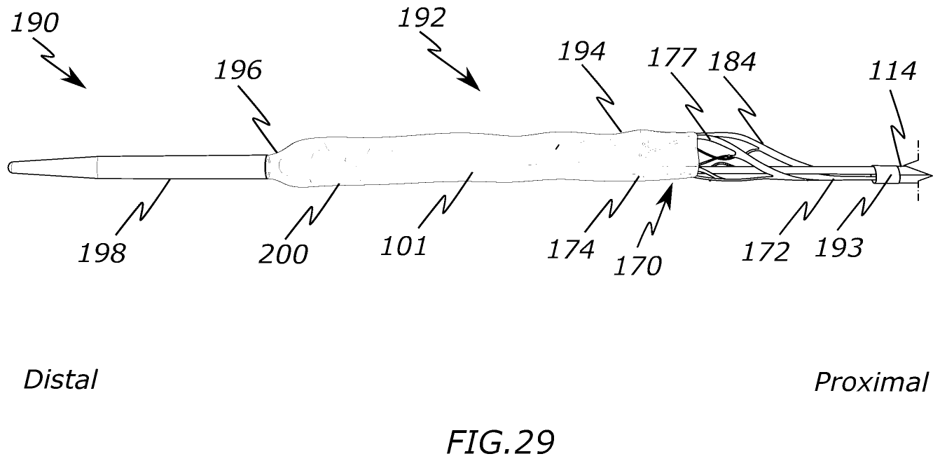

FIGS. 29-31 illustrate another example of a clot removal device 190. Also shown in FIGS. 29-31 are orientation indicators “Proximal” and “Distal.” FIGS. 29-31 are discussed below concurrently. The clot removal device 190 may include the elongated device body 101, a capture element 192, and the cutting element 170. In the examples of FIGS. 29-31, the elongated device body 101 may generally represent a tubular structure including a guidewire lumen extending therethrough. In some examples, the elongated device body 101 discussed with reference to the clot removal device 190 may be, or may be used in a similar manner, to the pusher 136 (FIGS. 9-10).

The cutting element 170 may be connected to the elongated device body 101. For example, the proximal portion 172 of the cutting element 170 may be clamped, welding, crimped, or otherwise secured to a distal end, portion, or region of the elongated device body 101, such as at location 193. The capture element 192 may be composed of a variety of polymeric layered, fiber, film, or matrix materials, such as including, but not limited to, nylon, polyether block amide (“Pebax”), polyethylene terephthalate (“PET”), polyurethane, polyester, or other materials. In some examples, the capture element 192 may be blood permeable to enable blood or other fluids to pass therethrough during a surgical operation. In such examples, the capture element 192 can be configured to suit a desired porosity of the clot removal device 190, such as by varying the thickness, density, or the number of individual material layers of the capture element 192 to vary the permeability or the porosity thereof.

The capture element 192 may include a proximal end 194 and a distal end 196. The proximal end 194 may be connected about a circumference of the distal portion 174 of the cutting element 170, such as, but not limited to, via adhesives or other chemical bonding means. For example, the proximal end 194 may be first wrapped or stretched circumferentially around an outer surface of the distal portion 174 and then glued thereto, or alternatively, the proximal end 194 may be applied circumferentially around an inner surface of the distal portion 174 and glued thereto.

In some examples, the distal end 196 of the capture element 192 may be connected to a distal tip 198 in a manner similar to the proximal end 194 (e.g., circumferentially bonded using adhesives), or may alternatively be clamped or otherwise secured within an interior of the distal tip 198. In some examples, the distal tip 198 may be a tube or a cap element forming various rounded or tapered shapes made from a metallic or polymeric material. The distal tip 198 may be secured to a distal end, portion, or region of the elongated device body 101, such as, but not limited to, via adhesives, clamping, and/or crimping.

When the proximal end 194 and the distal end 196 of the capture element 192 are connected to the distal tip 198 and to the cutting element 170, respectively, the capture element 192 may form a generally tubular shape that extends concentrically between with the distal portion 174 and/or the loop 177 and circumferentially encompasses the elongated device body 101. In this way, the capture element 192 can form a cavity 200 (e.g., and interior space) that faces, and is open and substantially aligned with, the proximal opening 191 of the loop 177. As may be appreciated, this may enable the capture element 192 to receive and retain clots, or pieces thereof, cut from a vessel wall by the one or more cutting surfaces 184 during proximal movement of the clot removal device 190.

FIGS. 29-31, along with FIGS. 20-21, show a progression of how the clot removal device 190 may be operated to cut and capture a clot (not shown). First, the clot removal device 190 may be positioned at a target location within vasculature of a patient in a variety of different ways, such as including, but not limited to, by following a guidewire (e.g., the elongated device body 101 may include a guidewire lumen extending between its proximal and distal end), by being advanced through a larger access catheter, or a combination thereof.

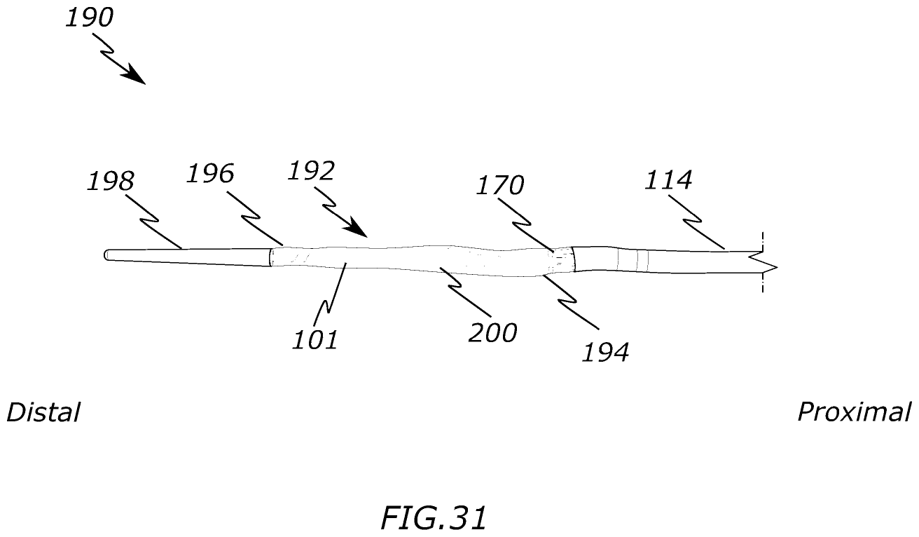

Next, a distal end, portion, or region of the clot removal device 190 may be advanced partially, or fully, beyond a clot. The outer sheath 114 may then be proximally withdrawn to allow the cutting element 170 to radially expand from a radially compressed configuration, such as shown in FIG. 31, to a radially expanded configuration, such as shown in FIGS. 20-21 and FIGS. 29-30, and, in turn, open and expand both the loop 177 and the cavity 200 of the capture element 192, by virtue of its connection to the distal portion 174 of the cutting element 170.

The elongated device body 101 may subsequently be moved proximally so that the one or more cutting surfaces 184 of the loop 177 cut the clot from the vessel wall, and at least a portion of the clot moves into the cavity 200 of the capture element 192 through the loop 177 and the distal portion 174. The cutting element 170 and the capture element 192 can then be partially, or fully, withdrawn into the outer sheath 114, such as shown in FIG. 31. Finally, the outer sheath 114 may be withdrawn from the patient, or alternatively, once the cavity 200 is located within in the outer sheath 114, the clot may be aspirated therefrom via one or more aspiration passages located within the outer sheath 114. As with all the examples described in this specification, multiple cutting and/or aspiration cycles or passes may be possible or desirable to retrieve or dissect a clot, such as during the removal of relatively long or large clots.

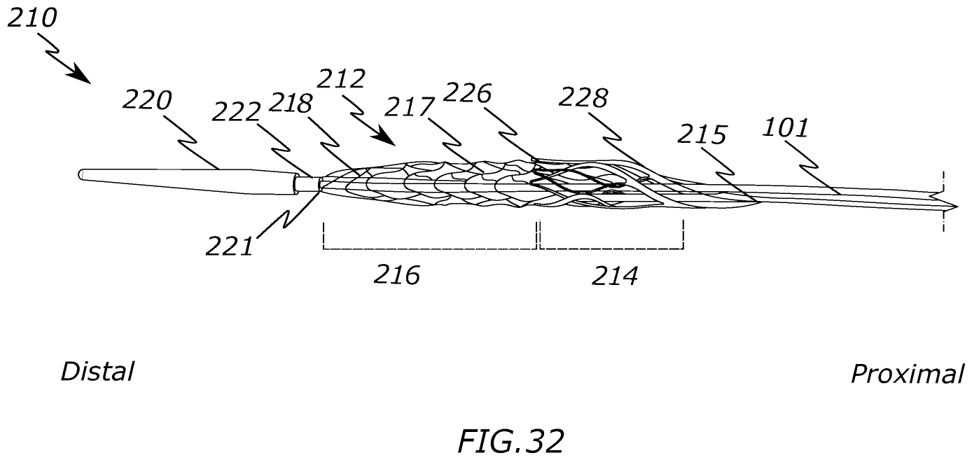

FIG. 32 illustrates another example clot removal device 210. The clot removal device 210 may include the elongated device body 101 and a cutting and capture element 212. In the examples described with reference to FIG. 32, the elongated device body 101 may generally represent a tubular structure including a guidewire lumen extending therethrough. In some examples, the elongated device body 101 discussed with reference to the clot removal device 210 may be, or may be used in a similar manner, to the pusher 136 (FIGS. 9-10).

The cutting and capture element 212 may include a proximal region 214 and a distal region 216. The proximal region 214 may be the cutting element of the clot removal device 210. In some examples, such as shown in FIG. 28, the proximal region 214 may represent a cutting element that is similar to the cutting element 170 described above. As such, the proximal region 214 may include any of the features or aspects discussed above with reference to the cutting element 170. The proximal region 214 may be connected to a distal end, portion, or region of the elongated device body 101, such as at location 215, via clamping, crimping, welding, adhesives, or other means or techniques. In some examples, the proximal region 214 may alternatively represent other cutting elements discussed in the present disclosure.

The distal region 216 may be a capture element or capture basket. In some examples, the distal region 216 may be a braided mesh structure, such as made from a plurality of interwoven wires, or a laser-cut mesh structure formed from a plurality of struts, forming a plurality of open cells 217. The distal region 216 may be made from a metallic shape memory material (e.g., Nitinol), a metallic non-shape memory material (e.g., stainless steel), or other materials, such as, but not limited to, a polymeric material. The mesh structure 218 may extend distally from the proximal region 214 to a distal tip 220. The mesh structure 218 may be connected to a distal tip 220, such as at location 221, via adhesives, welding, crimping, clamping, or other means or techniques. In some examples, the distal tip 220 may be a tube or a cap element forming various rounded or tapered shapes made from a metallic or polymeric material. The distal tip 220 may be secured to a distal end, portion, or region of the elongated device body 101, such as, but not limited to, via adhesives, clamping, and/or crimping. In further examples, the distal region 216 may also include one or more additional layers, such as an outer membrane layer similar to the capture element 192, either alternatively, or in addition to, the inner layer 222.

In some examples, the distal region 216 may be a single layer capture element or basket. For example, the mesh structure 218 may be used alone as the capture element of the clot removal device 210. In other examples, the distal region 216 may be a multiple layer capture element or basket. For example, the mesh structure 218 may be used as an outer layer, and an inner layer 224 may be located therein. The inner layer 222 may be similar to the capture element 192, at least in that the inner layer can be made from, but not limited to, a variety of polymeric layered, a film, matrix, or one or more layers of polymeric materials including, but not limited to, nylon, polyether block amide (“Pebax”), polyethylene terephthalate (“PET”), polyurethane, or polyester. In further examples, the distal region 216 may also include one or more additional layers, such as an outer membrane layer similar to the capture element 192, either alternatively, or in addition to, the inner layer 222.

The inner layer 222, similarly to the capture element 192, or one or more additional layers, such as an outer membrane layer, may also be connected to both the distal tip 220, such as at location 221, and a distal portion or end 226 of the proximal region 214. The inner layer 222 may also be blood permeable, such as to allow blood other fluids to pass therethrough. In such examples, the inner layer 222, or one or more additional layers, such as an outer membrane layer, can be configured to suit a desired porosity of the clot removal device 210, such as by varying the thickness, density, or the number of individual material layers of the inner layer 222, or the one or more additional layers, such as an outer membrane layer, to vary the permeability or the porosity thereof. As such, depending on whether the mesh structure 218 is used alone, or is used with the inner layer 222 or one or more additional layers, such as an outer membrane layer, a cavity 225 (e.g., an interior space) for receiving and retaining clots cut by one or more cutting surfaces 228 of the proximal region 214 can be defined by the mesh structure 218 or the inner layer 222. In view of the above, the clot removal device 210 may be used to cut and remove a clot in a manner similar to as described with reference to the clot removal device 190.

However, in contrast to the cutting element 170 and the capture element 192, the proximal region 214 and the mesh structure 218 of the distal region 216 can be formed integrally to create a unibody or unitary tubular (e.g., stent-like) capture and cutting element. In one such example, the proximal region 214 and the mesh structure 218 of the distal region 216 can be formed from a single piece of metallic or polymeric tubing stock. This may be advantageous, as such an integral construction may allow for a simplified manufacturing process and may provide a stronger bond between the cutting element (e.g., the proximal region 214) and the capture element (e.g., the mesh structure 218 of the distal region 216) of a clot removal or retrieval device.

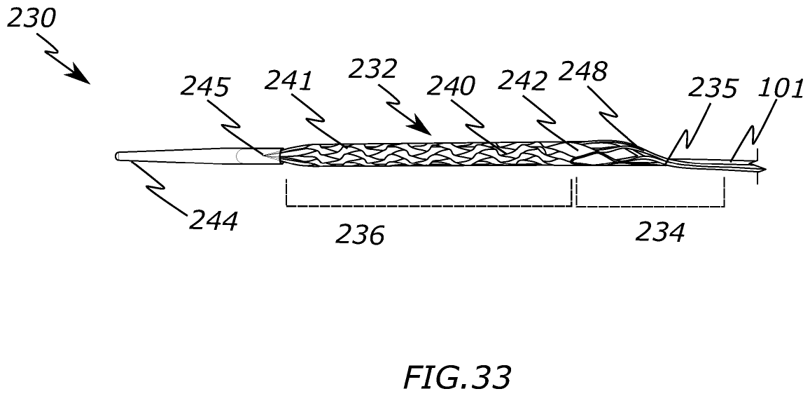

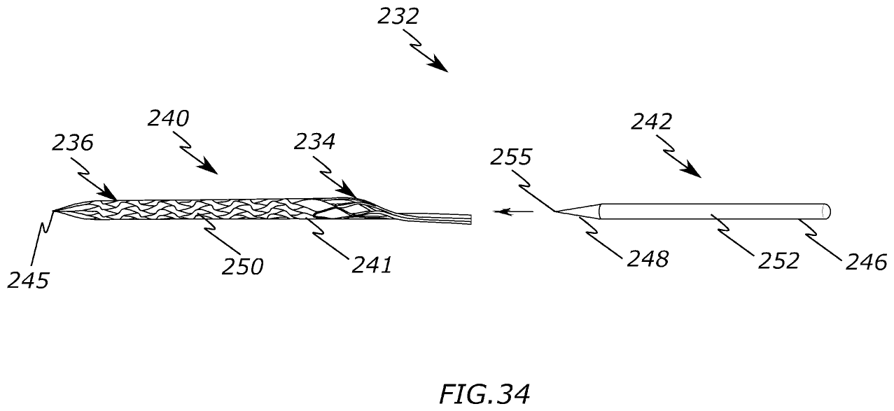

FIG. 33 illustrates another example clot removal device 230. FIG. 34 illustrates a cutting and capture element 232 of the clot removal device 230. Also shown in FIG. 33 are orientation indicators “Proximal” and “Distal”. FIGS. 33-34 are discussed below concurrently. In the examples described with reference to FIGS. 33-34, the elongated device body 101 may generally represent a tubular structure including a guidewire lumen extending therethrough. In some examples, the elongated device body 101 discussed with reference to the clot removal device 230 may be, or may be used in a similar manner, to the pusher 136 (FIGS. 9-10).

In some examples, such as shown in FIGS. 33-34, the proximal region 234 may represent a cutting element that is similar to the cutting element 170. As such, the proximal region 234 may include any of the features or aspects discussed above with reference to the cutting element 170. The proximal region 234 may be connected to a distal end, portion, or region of the elongated device body 101, such as at location 235, via clamping, crimping, welding, adhesives, or other means or techniques. In some examples, the proximal region 234 may alternatively represent other cutting elements discussed in the present disclosure.

The distal region 236 may be a capture basket or capture element. In some examples, the distal region 238 may include an outer component 240 and/or an inner component 242. The outer component 240 may be a braided mesh structure, such as made from a plurality of interwoven wires, or a laser-cut mesh structure formed from a plurality of struts, forming a plurality of open cells 241. The outer component 240 may be made from a metallic shape memory material (e.g., Nitinol), a metallic non-shape memory metallic material (e.g., stainless steel), or other materials, such as, but not limited to, a polymeric material. The outer component 240 may extend distally from the proximal region 234 to a distal tip 244. In further examples, the distal region 236 may also include one or more additional layers, such as an outer membrane layer that is similar to the capture element 192, either alternatively, or in addition to, the inner component 222.

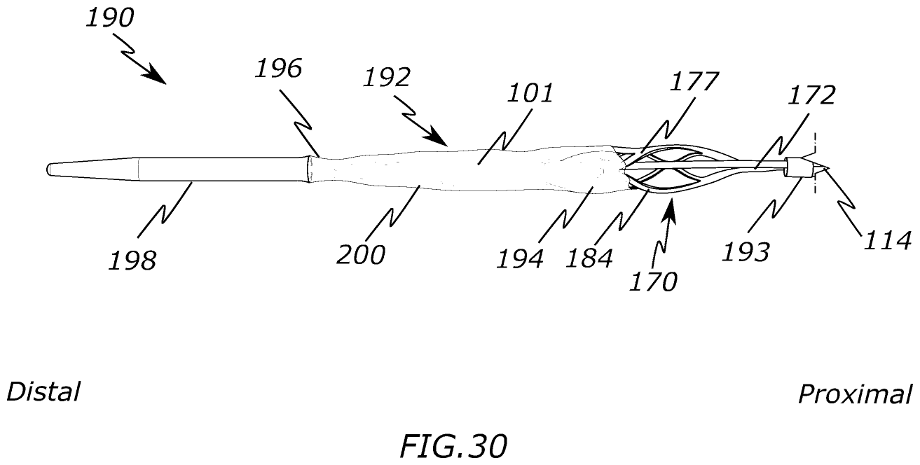

The outer component 240, and any of one or more additional layers extending therearound, such as an outer membrane layer, may converge, and be connected to, the distal tip 244 at location 245 there inside via adhesives, welding, crimping, clamping, or other means or techniques. The distal tip 244 may be a tube or a cap element forming various rounded or tapered shapes made from a metallic or polymeric material. The inner component 242 may be a tubular or otherwise hollow liner or insert receivable within a cavity 250 (e.g., an interior space) of the outer component 240, such as shown in FIG. 30. The inner component 242 can define an inner cavity 252 (e.g., an interior space) adapted to receive and retain a clot or cut or sheared portions thereof.

The inner component 242 can be adapted to contact and engage an inner surface or surface area of the inner component 242. For example, as shown in FIG. 34, the inner component 242 can include a cylindrical body portion 246 and a conical end portion 248. The cylindrical body portion 246 and the conical end portion 248 can each define outer surfaces or surface areas that are sized and shaped to conform or correspond to the cavity 250 of the outer component 240. The inner cavity 252 of the inner component 242 can be sealed at a distal end thereof, such as within the conical end portion 248 at point 255. The inner component 242 can be made from a variety of polymeric layered, fiber, film, or matrix materials, such as including, but not limited to, nylon, polyether block amide (“Pebax”), polyethylene terephthalate (“PET”), polyurethane, or polyester.

In some examples, the inner component 242 may also be blood permeable, such to allow blood or other fluids to pass therethrough. In some examples, the inner component 242 may be blood permeable to enable blood or other fluids to pass therethrough during a surgical operation. In such examples, the inner component 242 can be configured to suit a desired porosity of the clot removal device 230, such as by varying the thickness, density, or the number of individual material layers of the inner component 242 to vary the permeability or the porosity thereof.

Similar to the cutting and capture element 212, but in contrast to the cutting element 170 and the capture element 192, the proximal region 234 and the distal region 236 of the cutting and capture element 232 can be formed integrally to create a unibody or unitary tubular (e.g., stent-like) structure. In one such example, the proximal region 234 and the outer component 240 of the distal region 236 can be formed from a single piece of metallic or polymeric tubing stock. This may be advantageous, as such an integral construction may allow for a simplified manufacturing process and may provide a stronger bond between the cutting element (e.g., the proximal region 234) and at least a portion (e.g., the outer component 240 of the distal region 236) of the capture element of a clot removal or retrieval device.

Moreover, in contrast to the inner layer 222 of the cutting and capture element 212, the inner component 242 can be manufactured as a standalone structure that is not initially bonded or otherwise connected to the distal tip 244 of the proximal region 234. For example, the proximal region 234 and the distal region 236 may first be constructed, and the distal region 236 can be connected within the distal tip 244 at location 245. Next, the inner component 242 may subsequently be inserted into, and secured within, the cavity 250 of the outer component 240, such as, but not limited to, by applying an adhesive to outer surface or surface area of the inner component 242 and an inner surface or surface area of the outer component 240 within the cavity 250. In view of the above, the clot removal device 230 may be used to cut and capture a clot in a substantially similar manner to the clot removal device 190 and the clot removal device 210 previously described above. Any of the examples described in this specification above may also include one or more radiopaque markers or elements. For example, any or all of the attachment points (e.g., tubes, welds, adhesion points, or others) such as, but not limited to, points at or near the tip or clamp member 108, the distal tip 198, the distal tip 220, the distal tip 254, the location 112, the location 122, the location 124, the location 128, the location 193, the location 215, the location 221, the location 235, or the location 251, may include or be composed of radiopaque material. This may allow the physician to better visualize the position of the different components of the retrieval device relative to the clot.

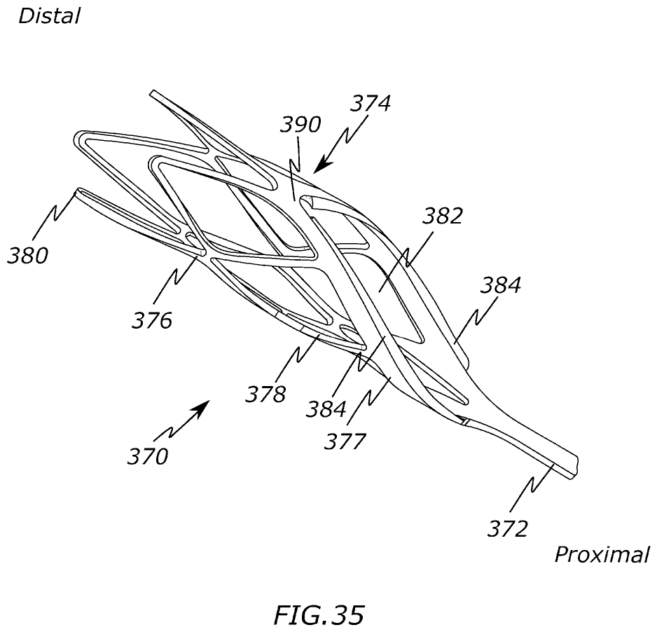

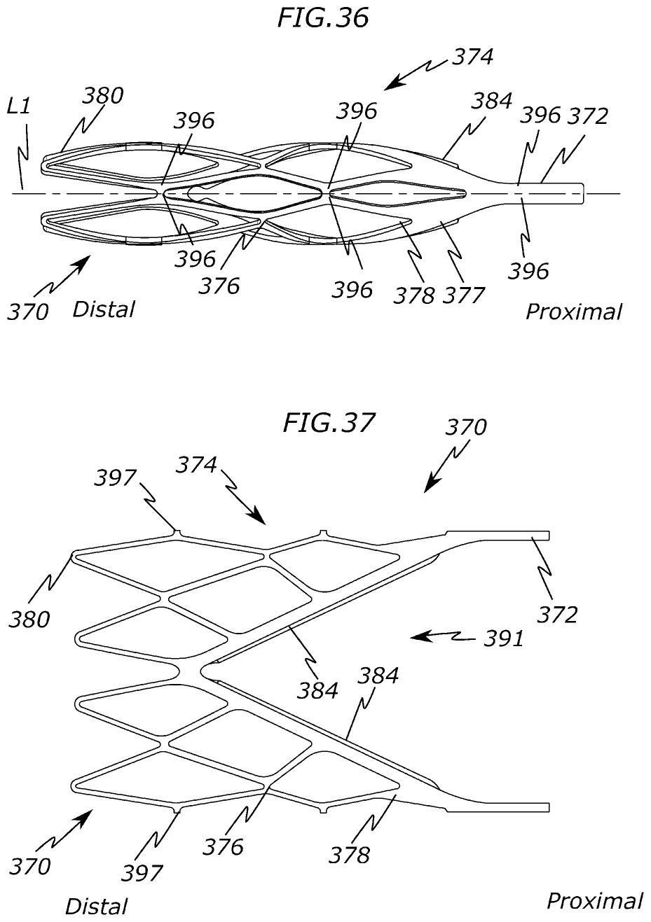

FIGS. 35-37 illustrate a cutting element 370 and are discussed below concurrently. In particular, FIG. 35 illustrates a side view of the cutting element 370 in a radially expanded configuration, FIG. 36 illustrates a bottom view of the cutting element 370 in the radially expanded configuration, and FIG. 37 illustrates a top view of the cutting element 370. FIGS. 35-37 also illustrate orientation indicators “Proximal” and “Distal.” The cutting element 370 may be similar to the cutting element 170, such as by including, among others, a proximal portion 372, a distal portion 374, a plurality of struts 376 each having a proximal portion 378 and distal portion 380, a loop 377, one or more cutting surfaces 384, and a proximal opening 391. While numbered differently than the cutting element 170, these elements are intended to be generally similar to those of the cutting element 170 (or other cutting elements and features described in this specification) unless otherwise noted.

However, in contrast to the one or more cutting surfaces 184 of the loop 177 of the cutting element 170, the one or more cutting surfaces 384 of the loop 377 may protrude inwardly and/or proximally outward from a proximal face 390 of the loop 377. In some examples, this may enable the one or more cutting surfaces 384 to form a variety of different, or additional shapes, than may otherwise be possible with cutting surfaces defined a cross-sectional shape of the loop 377. In some examples, the one or more cutting surfaces 384 may be representative of, or may include, one or more of the cutting elements shown in, and discussed with reference to, any of FIGS. 38-53 below.

The cutting element 370 may be constructed using a manufacturing technique that may be similar or different relative to, for example, the cutting element 170. For example, the cutting element 370 may be constructed with a process that allows for defining and sharpening the one or more cutting surfaces 384 before the loop 377 is forms a tubular or circular passage or opening therethrough. Such as process is shown in FIG. 35-37. First, as shown in FIG. 37, the cutting element 370 may be laser-cut, molded, machined, stamped, or otherwise shaped from a flat piece of material. At this stage, the one or more cutting surfaces 384 may further be sharped or otherwise prepared by grinding, laser cutting, polishing, and/or honing to a desired level or sharpness or edge shape.

Second, the cutting element 370 may be rolled, hammered, or otherwise bent radially around a cylindrical mandrel or other object to cause the distal portion 374 to form or circular shape, thereby creating the shape of the loop 377 and the proximal opening 391. Third, the cutting element 370 may be welded along a weld line L1, the path of which may include a length of the proximal portion 372 and/or at a plurality of weld points 396 of the distal portion 374. In some examples, the cutting element 370 may include a plurality of joining bosses 397 each adapted to be welded, joined, or otherwise affixed to an adjacent boss of the plurality of joining bosses 397. Finally, in some examples, the cutting element 370 may undergo further post processing operations, which may include griding or polishing of the plurality of weld points 396 to create a smoother surface, or heat treating of the cutting element 370 to relieve welding stresses, reduce or eliminate brittleness, or increase strength and ductility.

As previously discussed, the example devices described in this specification may include one or a plurality of cutting edges or surfaces. While the cutting edges described in this specification are generally shown with a generally uniform or straight edge, other edge shapes may also be possible, as further discussed below. In other words, any of the following cutting edges, elements, or surfaces may be used with any of the devices described in this specification.

FIG. 38 illustrates a side view of a first serrated cutting element 360, according to one example of the present disclosure, while FIG. 39 illustrates a magnified view of a portion of the first serrated cutting element 360. FIGS. 38-39 are discussed below concurrently.

The first serrated cutting element 360 may be defined by a plurality of concave surfaces 362 and a plurality of connecting surfaces 364. Each of the plurality of concave surfaces may form an inwardly curving semi-circular shape (e.g., a 180-degree arc). Each of the plurality of connecting surfaces 364 may form a peak or pointed shape. Each of the plurality of concave surfaces 362 may be similar or different to each other in size, shape, and/or curvature, and two adjacent concave surfaces of the plurality of concave surfaces 362 may be connected by a connecting surface of the plurality of connecting surfaces 364. In this way, the first serrated cutting element 360 may define a scalloped side profile.

In various examples, the plurality of concave surfaces 362 may include, but not limited to, one, two, three, four, five, six, seven, eight, nine, ten, or any greater number of individual concave surfaces, and the plurality of connecting surfaces 364 may include, but not limited to, two, three, four, five, six, seven, eight, nine, ten, eleven, or any greater number of individual connecting surfaces, respectively. As may be appreciated, the number of individual concave surfaces of the plurality of concave surfaces 362, and the number of individual connecting surfaces of the plurality of connecting surfaces 364, may each correspondingly vary depending upon the length of a cutting edge or surface that the first serrated cutting element 360 comprises, such as, but not limited to, the one or more cutting surfaces 184 of the loop 177, the one or more cutting surfaces 187 of the loop 179, or the one or more cutting surfaces 384 of the loop 377 described above.

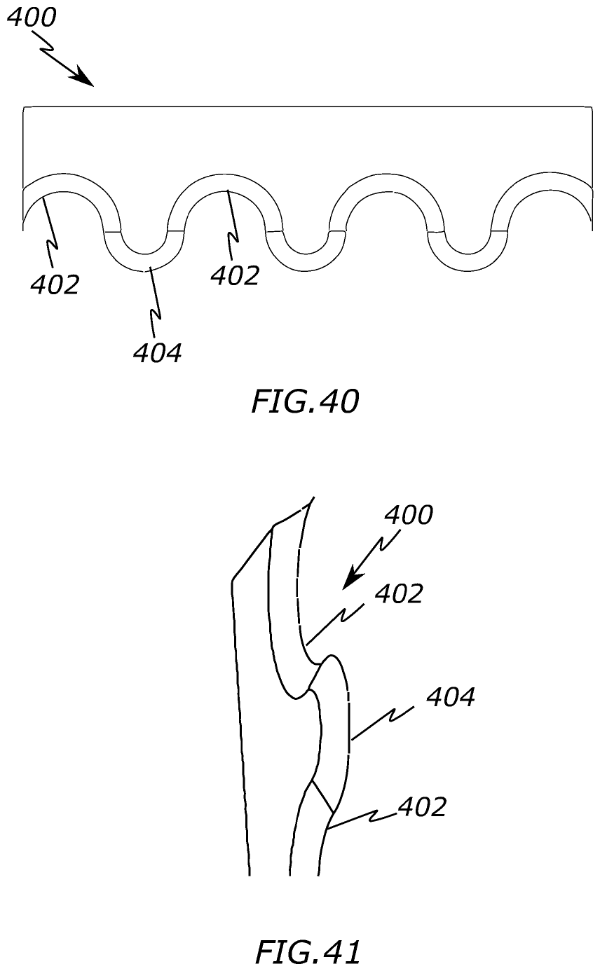

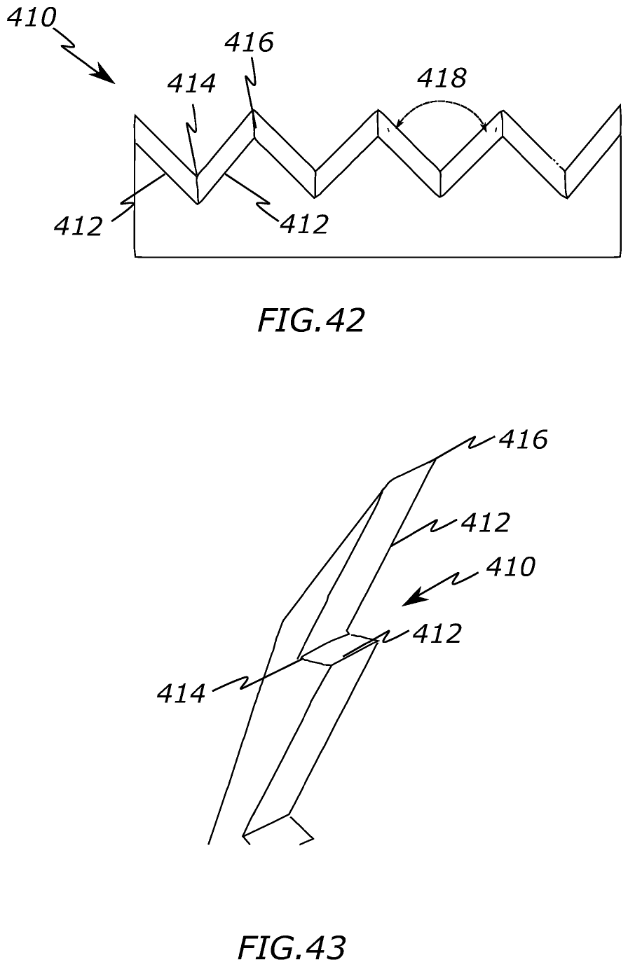

FIG. 40 illustrates a side view of a second serrated cutting element 400, according to one example of the present disclosure, while FIG. 41 illustrates a magnified view of a portion of the second serrated cutting element 400. FIGS. 41-42 are discussed below concurrently.

The second serrated cutting element 400 may be defined by a plurality of concave surfaces 402 and a plurality of convex surfaces 404. Each of the plurality of concave surfaces may form an inwardly curving shape semi-circular shape (e.g., a forward 180-degree arc), and each of the plurality of convex surfaces may form an outwardly curving semi-circular shape (e.g., a reverse 180-degree arc). Each of the plurality of concave surfaces 402 and each of the plurality of convex surfaces 404 may be similar or different to each other in size, shape, and/or curvature, and each of the plurality of concave surfaces 402 may either form an end of the second serrated cutting element 400 or be connected to a convex surface of the plurality of convex surfaces 404. In this way, the second serrated cutting element 400 may define a side profile having a repeating S-curve or wave shaped pattern.

In various examples, the plurality of concave surfaces 402 may include, but not limited to, one, two, three, four, five, six, seven, eight, nine, ten, or any greater number of individual concave surfaces 402, and the plurality of convex surfaces 404 may include, but not limited to, two, three, four, five, six, seven, eight, nine, ten, eleven, or any greater number of individual convex surfaces, respectively. As may be appreciated, the number of individual concave surfaces of the plurality of concave surfaces 402, and the number of individual connecting surfaces of the plurality of the plurality of convex surfaces 404, may each correspondingly vary depending upon the length of a cutting edge or surface that the second serrated cutting element 400 comprises, such as, but not limited to, the one or more cutting surfaces 184 of the loop 177, the one or more cutting surfaces 187 of the loop 179, or the one or more cutting surfaces 384 of the loop 377 described above.

FIG. 42 illustrates a side view of a third serrated cutting element 410, according to one example of the present disclosure, while FIG. 43 illustrates a magnified view of a portion of the third serrated cutting element 410. FIGS. 42-43 are discussed below concurrently.

The third serrated cutting element 410 may be defined by a plurality of pairs of angled surfaces 412, a plurality of inner connecting surfaces 414, and a plurality of outer connecting surfaces 416. Each of the plurality of inner connecting surfaces 414 and each of the plurality of outer connecting surfaces 416 may be a peak, pointed shape, or triangular shape formed at the intersection of a pair of the plurality of pairs of angled surfaces 412. Each pair of the plurality of pairs of angled surfaces 412 may form an angle 418 measured from an inner connecting surface located therebetween, that may be between about, but not limited to, 10 degrees and about 170 degrees. In one example, such as shown in FIG. 42, the angle 418 may be about 45 degrees.

In some examples, the angle 418 may be similar between one or more pairs of the plurality of pairs of angled surfaces 412, such as to form a symmetric serration pattern. In other examples, the angle 418 may be different between one or more pairs of the plurality of pairs of angled surfaces 412, such as to form a repeating or non-repeating asymmetric serration pattern. In view of the above, the third serrated cutting element 410 may define a side profile having a repeating V-shape or zigzag pattern.

In various examples, the plurality of inner connecting surfaces 414 may include, but not limited to, one, two, three, four, five, six, seven, eight, nine, ten, or any greater number of individual connecting surfaces, and the plurality of outer connecting surfaces 416 may include, but not limited to, two, three, four, five, six, seven, eight, nine, ten, eleven, or any greater number of individual convex surfaces, respectively. As may be appreciated, the number of individual surfaces of the plurality of inner connecting surfaces 414, and the number of individual connecting surfaces of the plurality of outer connecting surfaces 416, may each correspondingly vary depending upon the length of a cutting edge or surface that the third serrated cutting element 410 comprises, such as, but not limited to, the one or more cutting surfaces 184 of the loop 177, the one or more cutting surfaces 187 of the loop 179, or the one or more cutting surfaces 384 of the loop 377 described above.

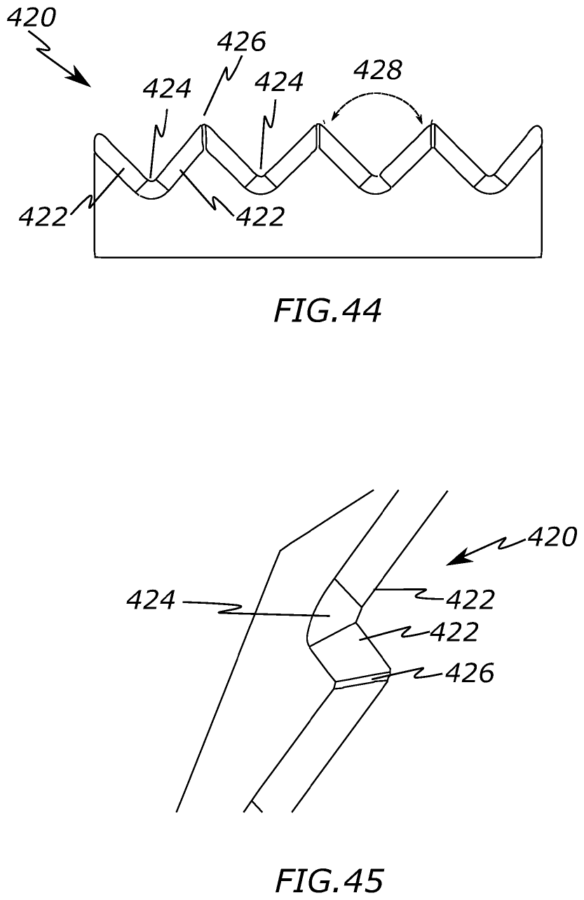

FIG. 44 illustrates a side view of a fourth serrated cutting element 420, according to one example of the present disclosure, while FIG. 45 illustrates a magnified view of a portion of the fourth serrated cutting element 420. FIGS. 44-45 are discussed below concurrently.

The fourth serrated cutting element 420 may be defined by a plurality of pairs of angled surfaces 422, a plurality of inner connecting surfaces 424, and a plurality of outer connecting surfaces 426. Each of the plurality of inner connecting surfaces 424 and/or each of the plurality of outer connecting surfaces 426 may be a curved surface or peaked, pointed, or triangular shapes connecting a pair of the plurality of pairs of angled surfaces 422.

Each pair of the plurality of angled surfaces 422 may form an angle 428 measured from a center point of an inner connecting surface 424 located therebetween, that may be between about, but not limited to, 10 degrees and about 170 degrees. In one example, such as shown in FIG. 44, the angle 428 may be about 90 degrees. In some examples, the angle 428 may be similar between one or more pairs of the plurality of pairs of angled surfaces 422, such as to form a symmetric serration pattern. In other examples, the angle 428 may be different between one or more pairs of the plurality of pairs of angled surfaces 422, such as to form a repeating or non-repeating asymmetric serration pattern.

In various examples, the plurality of inner connecting surfaces 424 may include, but are not limited to, one, two, three, four, five, six, seven, eight, nine, ten, or any greater number of individual connecting surfaces, and the plurality of outer connecting surfaces 426 may include, but not limited to, two, three, four, five, six, seven, eight, nine, ten, eleven, or any greater number of individual convex surfaces, respectively. As may be appreciated, the number of individual surfaces of the plurality of inner connecting surfaces 424, and the number of individual connecting surfaces of the plurality of outer connecting surfaces 426, may each correspondingly vary depending upon the length of a cutting edge or surface that the fourth serrated cutting element 420 comprises, such as, but not limited to, the one or more cutting surfaces 184 of the loop 177, the one or more cutting surfaces 187 of the loop 179, or the one or more cutting surfaces 384 of the loop 377 described above.

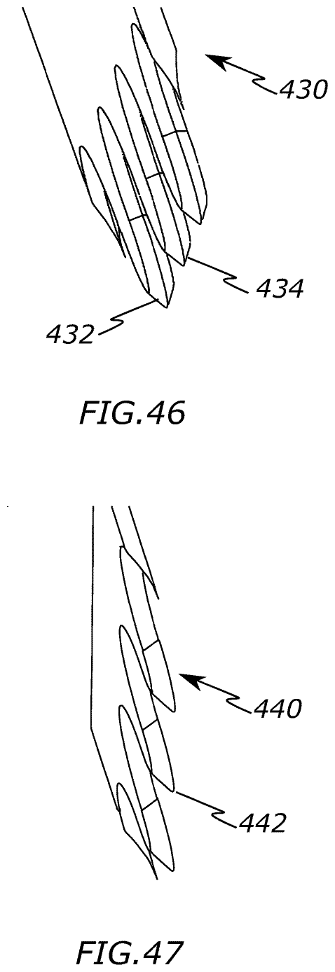

FIG. 46 illustrates a perspective view of a cutting element 430 defining a pair of sharped edges including a first sharpened edge 432 and a second sharped edge 434. The cutting element 430 may generally represent how any of the serrated cutting elements discussed with reference to FIGS. 38-45 may be a double-edged serrated cutting element (i.e., an angled surface on opposite sides of the cutting element). For example, the plurality of concave surfaces 362 and the plurality of connecting surfaces 364 of the first serrated cutting element 360, the plurality of concave surfaces 402 and the plurality of convex surfaces 404 of the second serrated cutting element 400, the plurality of pairs of angled surfaces 412, the plurality of inner connecting surfaces 414, the plurality of outer connecting surfaces 416 of the third serrated cutting element 410, and the plurality of pairs of angled surfaces 422, the plurality of inner connecting surfaces 424, and the plurality of outer connecting surfaces 426 of the fourth serrated cutting element 420 may define a generally triangular cross-sectional shape, such as illustrated in any of FIGS. 49-52. Put another way, surfaces and shapes facing the viewer in FIGS. 38, 40, 42, and 44 may be the same on the opposite side of the cutting element that is hidden from view on its opposite side.

FIG. 47 illustrates a cutting element 440 defining a single sharpened edge 442. The cutting element 440 may generally represent how any of the serrated cutting elements discussed with reference to FIGS. 38-45 may be a single-edged serrated cutting element. For example, the plurality of concave surfaces 362 and the plurality of connecting surfaces 364 of the first serrated cutting element 360, the plurality of concave surfaces 402 and the plurality of convex surfaces 404 of the second serrated cutting element 400, the plurality of pairs of angled surfaces 412, the plurality of inner connecting surfaces 414, the plurality of outer connecting surfaces 416 of the third serrated cutting element 410, and the plurality of pairs of angled surfaces 422, the plurality of inner connecting surfaces 424, and the plurality of outer connecting surfaces 426 of the fourth serrated cutting element 420 may define a generally angled or sloped cross-sectional shape, such as illustrated in FIG. 53.

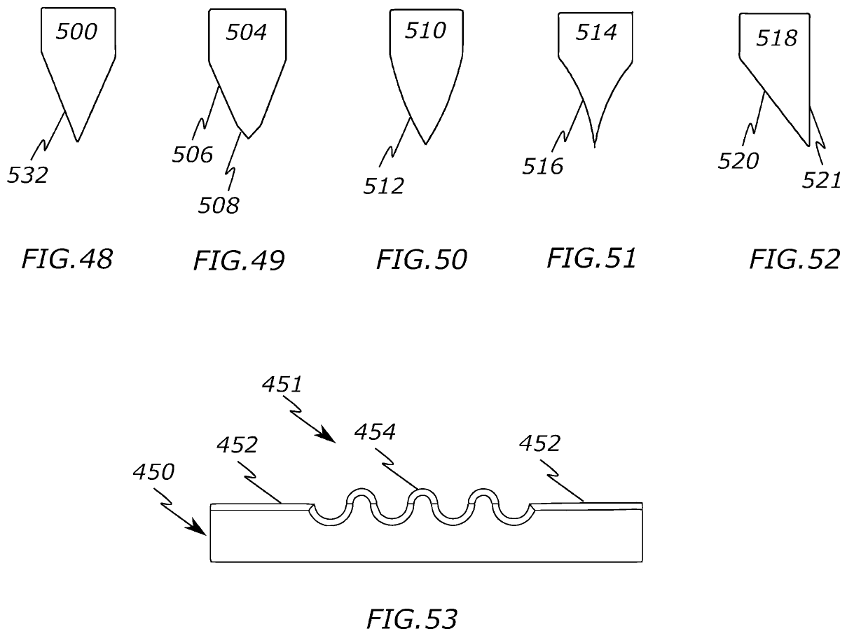

FIG. 48 illustrates a cross-section of a double-edged cutting element 500. The double-edged cutting element 500 may form a partially rectangular cross-section within a pointed or triangular end. As may be appreciated, the first pair of cutting edges 532 may form various acute angles relative to each other at the pointed or triangular end.

FIG. 49 illustrates a cross-section of a double-bevel cutting element 504. The double-bevel cutting element 504 may have form a rectangular cross-section with a pointed double-beveled end. In contrast to the double-edged cutting element 500, the double-bevel cutting element 504 may include a first pair of cutting edges 506 and a second pair of cutting edges 508. As may be appreciated, each of the first pair of cutting edges 506 may be located or defined at various obtuse angles relative to an adjacent cutting edge of the second pair of cutting edges 508.

FIG. 50 illustrates a convex cutting element 510 having a pair of outwardly curved cutting edges 512. The convex cutting element 510 may form a rectangular cross-section having a pointed end. As may be appreciated, the pair of outwardly curved cutting edges 512 may form various acute or obtuse angles relative to each other at the pointed end, such as depending upon a curvature or arc of each of the outwardly curved cutting edges 512.

FIG. 51 illustrates a concave cutting element 514 having a pair of inwardly curved cutting edges 516. The concave cutting element 514 may form a rectangular cross-section having a pointed end. As may be appreciated, the pair of inwardly curved cutting edges 516 may form various acute or obtuse angles relative to each other at the pointed end, such as depending upon a curvature or arc of each of the inwardly curved cutting edges 516.

FIG. 52 illustrates a single-edged cutting element 518 having an angled cutting edge 520. The single-edged cutting element 518 may form a rectangular cross-section having a pointed end that is offset from a centerline of the single-edged cutting element 518. As may be appreciated, the angled cutting edge 520 may form various acute angles relative to an adjacent surface 521, such as depending upon a curvature or arc of each of the inwardly curved cutting edges 516. Further, in some examples, the angled cutting edge 520 may be planar surface, or may alternatively be an inwardly curved (e.g., concave), outwardly curved (e.g., convex), bevel, or any other surface including varied geometry.

FIG. 53 illustrates a side view of a combination cutting element 450. A combined cutting surface 451 of the combination cutting element 450 may be generally representative of, or may include any repeating or non-repeating combination or mix of, the first serrated cutting element 360, the second serrated cutting element 400, the third serrated cutting element 410, the fourth serrated cutting element 420, or other types, shapes, styles, or edges that may be combined along a single cutting element, such as, but not limited to, the loop 177, the loop 179, or the loop 377 described above. In one example, such as shown in FIG. 48, the combined cutting surface 451 may be a single-edged cutting element including a linear cutting surface 452 adjacent to a serrated cutting surface 454. In other examples, the combined cutting surface 451 may include two or more different serrated cutting edges or shapes, such as any of the edges or shapes shown in FIGS. 38-47 above. In one example, the linear cutting surface 452 and serrated cutting surface 454 form an alternating pattern. In another example, the linear cutting surface 452 or serrated cutting surface 454 may be located only at certain locations, such only on a distal-most portion of the device while the other cutting surface may be located on more proximal locations.

### Claim Bank

Clause 1. A clot removal device, comprising: an elongated device body having a distal region; a cutting element located at the distal region of the elongated device body; and, a capture element located at the distal region of the elongated device body; wherein the capture element has a radially compressed shape and a radially expanded shape forming a cavity having an opening into the cavity.

Clause 2. The clot removal device of clause 1, wherein the cutting element comprises a wire or ribbon having at least one edge extending along at least part of the cutting element.

Clause 3. The clot removal device of clause 1, wherein the elongated device body comprises an elongated inner catheter member positioned within a lumen of an elongated outer catheter member; and wherein the elongated inner catheter member can move relative to the elongated outer catheter member.

Clause 4. The clot removal device of clause 3, wherein the capture element is connected to a distal region of the elongated inner catheter member and the cavity of the capture element opens in a proximal direction; and wherein the cutting element is connected between a distal region of the elongated outer catheter member and the capture element.

Clause 5. The clot removal device of clause 4, wherein the cutting element is one or a plurality of wires; and wherein the wires comprise one or more sharpened edges.

Clause 6. The clot removal device of clause 3, wherein the capture element is connected to a distal region of the outer catheter member and the cavity of the capture element opens in a distal direction.

Clause 7. The clot removal device of clause 6, wherein the cutting element is connected to the distal region of the outer catheter member and to a distal region of the inner catheter member; wherein the cutting element comprises one or a plurality of wires with sharpened edges.

Clause 8. The clot removal device of clause 7, further comprising a radially expandable closure member; wherein the expandable closure member has an expanded configuration sized to block the opening of the cavity.

Clause 9. The clot removal device of clause 8, wherein the outer catheter member includes a proximal aspiration port for connection to an aspiration source.

Clause 10. The clot removal device of clause 1, wherein the capture element is a mesh basket and the opening into the cavity is proximally-facing.

Clause 11. The clot removal device of clause 10, wherein the cutting element is disposed around at least part of an edge of the opening into the cavity.

Clause 12. A clot removal device, comprising: an elongated device body having an elongated inner catheter member positioned within a lumen of an elongated outer catheter member; and wherein the elongated inner catheter member is movable relative to the elongated outer catheter member; at least one wire connected at a distal region of the elongated inner catheter member and at a distal region of the elongated outer catheter member; and, an expandable capture basket having a radially compressed shape and a radially expanded shape forming a cavity sized to capture a clot.

Clause 13. The clot removal device of clause 12, wherein the at least one wire has an expanded shape extending away from the elongated inner member.

Clause 14. The clot removal device of clause 13, wherein the at least one wire is connected to the expandable capture basket such that when the elongated inner catheter member is moved distally relative to the elongated outer catheter member, the at least one wire substantially closes a proximally-facing opening of the cavity.

Clause 15. The clot removal device of clause 14, wherein the at least one wire helically encircles the elongated inner member.

Clause 16. The clot removal device of clause 13, wherein a proximal portion of the at least one wire is positioned within the cavity of the expandable capture basket and is positioned out a distally-facing opening of the cavity.

Clause 17. The clot removal device of clause 16, further comprising a mesh shield having a radially expanded shape; and wherein the elongated inner member is proximally movable to move the mesh shield adjacent to the distally-facing opening of the cavity.

Clause 18. The clot removal device of clause 12, further comprising a first radiopaque marker at the distal region of the elongated inner catheter member and a second radiopaque marker at the distal region of the elongated outer catheter member.

Clause 19. The clot removal device of clause 17, wherein the elongated inner catheter member is rotatable relative to the elongated outer catheter member such that the at least one wire is wrapped or unwrapped around the elongated inner catheter member.

Clause 20. A clot removal device, comprising: an elongated device body; a cutting element means for cutting a clot; and, a capture element means for capturing a clot after the cutting element means has cut the clot.

Clause 21. A clot removal device, comprising: an elongated device body; a cutting element connected at a distal end of the elongated device body; the cutting element comprising a plurality of struts connected together in a tubular shape that has a radially compressed configuration and a radially expanded configuration; wherein at least some of the plurality of struts have one or more cutting surfaces at a proximal portion of the cutting element.

Clause 22. The clot removal device of clause 21, wherein at least some of the plurality of struts are connected together in a V shape or an alternating wave shape.

Clause 23. The clot removal device of clause 21, wherein at least some of the plurality of struts are tapered in width, where the width of the at least some of the plurality of struts is relatively wider 1) at a proximal region of the cutting element, 2) at a distal region of the cutting element, 3) at a middle region of the cutting element, or 4) at the proximal region and the distal region of the cutting element relative to the middle region of the cutting element.

Clause 24. The clot removal device of clause 21, wherein the cutting surfaces are scalloped, angled, or sloped regions along a strut.

Clause 25. The clot removal device of clause 21, further comprising a wire connected to the elongated device body and to a proximal portion of the cutting element.

Clause 26. The clot removal device of clause 25, wherein the wire is connected via one or more of the following: 1) through apertures in at least some of the plurality of struts, 2) by being tied around at least some of the plurality of struts, 3) by being welded to at least some of the plurality of struts, or 4) by being adhered with adhesive to at least some of the plurality of struts.

Clause 27. The clot removal device of clause 21, further comprising a capture element connected to the cutting element.

Clause 28. The clot removal device of clause 27, wherein the capture element is connected to a distal portion of the cutting element.

Clause 29. The clot removal device of clause 28, wherein the capture element is a basket comprising a braided mesh or a membrane.

Clause 30. A clot removal device, comprising: an elongated device body; and, a cutting element connected at a distal end of the elongated device body; the cutting element comprising a tubular shape that has a radially compressed configuration and a radially expanded configuration; wherein a proximal end of the tubular shape comprises a loop positioned at a biased angle within an inclusive range of 90 degrees to 180 degrees relative to a longitudinal axis of the tubular shape; and wherein the loop comprises one or more cutting surfaces.

Clause 31. The clot removal device of clause 30, wherein the loop is a closed loop or an open loop.

Clause 32. The clot removal device of clause 31, wherein the one or more cutting surfaces are located along one or more of 1) an inner surface of the loop, 2) an outer surface of the loop, or 3) a side surface of the loop.

Clause 33. The clot removal device of clause 31, wherein the one or more cutting surfaces are positioned at a single angle relative to the longitudinal axis of the tubular shape or at multiple angles relative to the longitudinal axis of the tubular shape.

Clause 34. The clot removal device of clause 31, wherein the one or more cutting surfaces are smooth or are serrated.

Clause 35. The clot removal device of clause 31, wherein the tubular shape further comprises a plurality of struts connected distally to the loop and that form a plurality of cells.

Clause 36. The clot removal device of clause 31, further comprising an elongated strut extending proximally from the loop.

Clause 37. The clot removal device of clause 31, further comprising an elongated strut extending proximally from a distal-most position of the loop.

Clause 38. A clot removal device, comprising: an elongated device body; a cutting element located at a distal region of the elongated device body; the cutting element comprising a tubular shape that has a radially compressed configuration and a radially expanded configuration; the cutting element having a proximal region including one or more cutting surfaces; a capture element located distally of the cutting element and forming a cavity opening towards the cutting element; and, a distal tip located distally of the capture element.

Clause 39. The clot removal device of clause 38, wherein the distal tip is connected to a distal end of the elongated device body.

Clause 40. The clot removal device of clause 39, further comprising a guidewire lumen extending through the elongated device body and through the distal tip.

Clause 41. The clot removal device of clause 39, wherein the capture element is connected to the cutting element and to the distal tip.

Clause 42. The clot removal device of clause 38, wherein the capture element is a polymer film or a polymer fabric forming the cavity.

Clause 43. The clot removal device of clause 42, wherein the polymer film or polymer fabric are composed of nylon, Pebax, PET, or polyurethane.

Clause 44. The clot removal device of clause 42, wherein the polymer film or polymer fabric are blood permeable.

Clause 45. The clot removal device of clause 42, wherein the capture element further comprises a braided or laser-cut structure positioned around the polymer film or polymer fabric.

Clause 46. A clot removal device, comprising: an elongated device body; and, a cutting and capture element located at a distal region of the elongated device body; the cutting and capture element comprising a tubular shape that has a radially compressed configuration and a radially expanded configuration; wherein the tubular shape has a proximal portion comprising a proximal opening and one or more cutting surfaces; and wherein the tubular shape has a distal portion forming a cavity with a plurality of blood-permeable openings.

Clause 47. The clot removal device of clause 46, wherein the proximal portion of the tubular shape comprises a braided or laser-cut structure forming a plurality of open cells.

Clause 48. The clot removal device of clause 47, wherein the cavity is further formed by a layer of 1) a film, 2) a fabric, or 3) a braided mesh.

Clause 49. The clot removal device of clause 48, wherein the film, the fabric, or the braided mesh are composed of polyester.

Clause 50. The clot removal device of clause 48, wherein the film, the fabric, or the braided mesh are located on an inside of the proximal portion of the tubular shape or on an outside of the proximal portion of the tubular shape.

1-50. (canceled)

51. A clot removal device, comprising:
an elongated device body;
a cutting element connected at a distal end of the elongated device body; the cutting element including a loop having one or more sharpened cutting surfaces that are oriented proximally.

52. The clot removal device of claim 51, wherein the cutting element further comprises a plurality of struts connected together in a tubular shape; and wherein the cutting element has a radially compressed configuration and a radially expanded configuration.

53. The clot removal device of claim 52, wherein, when the cutting element is in the radially compressed configuration, a proximally facing opening of the loop is positioned at a biased angle within an inclusive range of 90 degrees to 180 degrees relative to a longitudinal axis of the tubular shape.

54. The clot removal device of claim 51, wherein the loop is a complete loop or a partial loop; and wherein the one or more sharpened cutting surfaces are scalloped, angled, or tapered relative to other portions of the loop.

55. The clot removal device of claim 51, wherein the one or more sharpened cutting surfaces form an angle between an interior surface of the cutting element and an exterior surface of the cutting element that is within an inclusive range of about 10 degrees and about 35 degrees.

56. The clot removal device of claim 51, further comprising a capture element connected distally of the cutting element and forming a cavity opening towards the cutting element.

57. The clot removal device of claim 56, wherein the capture element is connected to a distal end of the cutting element and to a distal tip of the elongated device body.

58. The clot removal device of claim 56, wherein the capture element comprises a braided structure or a laser-cut structure; and wherein a film layer, a fabric layer, or a mesh layer is located on an inside or an outside of the braided structure or the laser-cut structure.

59. The clot removal device of claim 51, further comprising an elongated proximal strut extending proximally from the loop; the elongated proximal strut having a curved cross-sectional profile.

60. The clot removal device of claim 51, wherein the loop and the one or more sharpened cutting surfaces may comprise one or more of: a partially rectangular cross-section with a pointed or triangular end, a rectangular cross-section with a pointed double-beveled end, a rectangular cross-section with a pointed end and inwardly curved concave surfaces, and a rectangular cross-section with a pointed end offset from a centerline.

61. The clot removal device of claim 60, wherein the one or more sharpened cutting surfaces may comprise a linear cutting surface and a serrated cutting surface arranged in an alternating pattern.

62. The clot removal device of claim 52, wherein at least some of the plurality of struts are connected together in a V shape or an alternating wave shape.

63. The clot removal device of claim 52, wherein at least some of the plurality of struts are tapered in width, where the width of the at least some of the plurality of struts is relatively wider 1) at a proximal region of the cutting element, 2) at a distal region of the cutting element, 3) at a middle region of the cutting element, or 4) at the proximal region and the distal region of the cutting element relative to the middle region of the cutting element.

64. The clot removal device of claim 52, further comprising a wire connected to the elongated device body and to a proximal portion of the cutting element; wherein the wire is connected via one or more of the following: 1) through apertures in at least some of the plurality of struts, 2) by being tied around at least some of the plurality of struts, 3) by being welded to at least some of the plurality of struts, or 4) by being adhered with adhesive to at least some of the plurality of struts.

65. The clot removal device of claim 53, wherein the one or more sharpened cutting surfaces form an angle between an interior surface of the cutting element and an exterior surface of the cutting element that within an inclusive range of about 10 degrees and about 35 degrees; and further comprising a capture element connected distally of the cutting element and forming a cavity opening towards the cutting element; wherein the capture element is connected to a distal end of the cutting element and to a distal tip of the elongated device body.

66. A clot removal device, comprising:
an elongated device body;
a cutting element connected at a distal portion of the elongated device body; the cutting element including a loop having one or more sharpened cutting surfaces that are oriented proximally;
wherein the one or more sharpened cutting surfaces form an angle between either an interior surface of the cutting element or between an exterior surface of the cutting element that is within an inclusive range of about 10 degrees and about 35 degrees.

67. The clot removal device of claim 66, wherein the cutting element further comprises a plurality of struts connected together in a tubular shape; and wherein the cutting element has a radially compressed configuration and a radially expanded configuration.

68. The clot removal device of claim 67, wherein a proximally facing opening of the loop is positioned at a biased angle within an inclusive range of 90 degrees to 180 degrees relative to a longitudinal axis of the tubular shape.

69. The clot removal device of claim 68, wherein the loop is a complete loop or a partial loop; and wherein the one or more sharpened cutting surfaces are scalloped, angled, or tapered relative to other portions of the loop.

70. A clot removal device, comprising:
an elongated device body;
a cutting element connected at a distal end of the elongated device body; the cutting element including a loop having one or more regions for cutting a clot.
